(19) United States
(12) Patent Application Publication
(10) Pub. No.: US 2024/0374681 A1
(43) Pub. Date: Nov. 14, 2024

(54) Formulation for Co-Administration of Q-GRFT and Tenofovir

(71) Applicant: University of Pittsburgh - Of the Commonwealth System of Higher Education, Pittsburgh, PA (US)

(72) Inventors: Lisa Cencia Rohan, Pittsburgh, PA (US); Xin Tong, Lansdale, PA (US); Lin Wang, Pittsburgh, PA (US)

(21) Appl. No.: 18/688,651

(22) PCT Filed: Sep. 2, 2022
(86) PCT No.: PCT/IB2022/058289

§ 371 Date: Mar. 1, 2024

Related U.S. Application Data
(60) Provisional application No. 63/240,519, filed on Sep. 3, 2021.
(60) Provisional application No. 63/247,470, filed on Sep. 23, 2021.

(51) Int. Cl. A61K 38/16; A61K 9/00; A61K 9/19; A61K 31/675; A61K 47/26

(52) U.S. Cl. CPC A61K 38/168; A61K 9/0031; A61K 9/19; A61K 31/675; A61K 47/26

(57) ABSTRACT

Provided herein are stable hypotonic or isotonic formulations containing active ingredients, such as antiviral compositions, or anti-retroviral compositions for intrarectal delivery to provide prophylaxis against viral infections.

### CROSS-REFERENCE TO RELATED APPLICATIONS

This application is the United States national phase of International Application No. PCT/IB22/58289 filed Sep. 2, 2022, and claims the benefit of U.S. Provisional Patent Application No. 63/240,519, filed Sep. 3, 2021, and of U.S. Provisional Patent Application No. 63/247,470, filed Sep. 23, 2021, the disclosures of which are hereby incorporated by reference in their entireties.

### STATEMENT REGARDING FEDERALLY SPONSORED RESEARCH

This invention was made with government support under AI113182 awarded by the National Institutes of Health. The government has certain rights in the invention.

### BACKGROUND OF THE INVENTION

Douching/enema use is a common procedure which men who have sex with men (MSM) perform before intercourse. However, it can also be a risk factor for HIV/STI. Studies suggest that incorporating prevention strategies for human immunodeficiency virus (HIV) or sexually transmitted infection (STI) into this behavior could provide an optimal means for drug administration. Accordingly, there is a need in the field for improved formulations for preventing/treating STIs.

### SUMMARY OF THE INVENTION

Provided herein are two hypotonic enema formulations, each containing a combination of two anti-HIV drugs (Tenofovir (TFV) and Q-Griffithsin (Q-GRFT)), which were developed as a novel Pre-Exposure Prophylaxis (PrEP) option. These formulations offer dual protection against HIV/STI, benefitting from the synergetic effects of TFV and Q-GRFT. Further provided is an easy, robust, and reproducible development procedure to manufacture the final products, which are stable over time.

In one aspect or embodiment of the subject disclosed herein, a formulation, such as a hypotonic formulation, optionally contained within a rectal delivery device, is provided. The formulation comprises a therapeutic composition and a griffithsin protein in a composition comprising an excipient and a buffer. In some embodiments, the therapeutic composition is an antiretroviral composition, a nucleoside reverse transcriptase inhibitor, or a nonnucleoside reverse transcriptase inhibitor. In some embodiments, the therapeutic composition comprises tenofovir or a pharmaceutically-acceptable salt thereof. In some embodiments, the excipient is a disaccharide or sugar polyol, for example, maltitol, lactose, glucose, sorbitol, sucrose or trehalose.

In another aspect or embodiment, a method of producing a formulation, such as a hypotonic formulation, is provided. In some embodiments, the method comprises diluting a buffer and lowering the osmolality to form a first premix; adding a therapeutic composition to the first premix and mixing until the therapeutic composition is completely dissolved to form a second premix; adjusting the osmolality or the pH of the second premix; adding the griffithsin protein to the second premix under constant mixing to form a mixture, testing the pH or osmolality of the mixture and, if necessary, adjusting the pH to between 6.5-8 or adjusting the osmolality to between 123 mOsm/kg and 167 mOsm/kg; and adding the mixture to a rectal delivery device.

In another aspect or embodiment, a method of treating or preventing a sexually transmitted infection is provided. In some embodiments, the method comprises intrarectally delivering a formulation, as described herein, to a patient in a dosage regimen effective to treat or prevent a sexually transmitted infection.

In another aspect or embodiment, a method of providing prophylactic protection from human immunodeficiency virus is provided. In some embodiments, the method comprises placing the formulation, as described herein, intrarectally or orally in a patient.

In another aspect or embodiment, a rectal dosage form is provided. In some embodiments, the rectal dosage form comprises the hypotonic formulation, as described herein, contained in a rectal delivery device. In some embodiments, the rectal dosage form is configured in a liquid dosage form, a solid dosage form, or a semi-solid dosage form.

In another aspect or embodiment, a method of producing a stable Q-GRFT lyophilized powder is provided. The method comprises dissolving the Q-GRFT lyophilized with a disaccharide or sugar polyol, for example, maltitol, lactose, glucose, sorbitol, sucrose or trehalose, in an aqueous solvent, followed by drying, lyophilizing, or spray-drying the Q-GRFT-containing mixture.

Various aspects of the present disclosure may be further characterized by one or more of the following clauses:

Clause 1: A formulation comprising a therapeutic composition and a griffithsin protein in a composition comprising an excipient and a buffer, wherein the formulation is optionally hypotonic.

Clause 2: The formulation of clause 1, wherein the therapeutic composition is an antiretroviral composition.

Clause 3: The formulation of clause 1 or clause 2, wherein the therapeutic composition is a nucleoside reverse transcriptase inhibitor or a nonnucleoside reverse transcriptase inhibitor.

Clause 4: The formulation of any of clauses 1-3, wherein the therapeutic composition comprises tenofovir or a pharmaceutically-acceptable salt thereof.

Clause 5: The formulation of any of clauses 1-4, wherein the therapeutic composition is tenofovir.

Clause 6: The formulation of any of clauses 1-5, wherein the therapeutic composition is tenofovir alefanamide or tenofovir disoproxil.

Clause 7: The formulation of any of clauses 1-6, wherein the therapeutic composition is tenofovir alefanamide.

Clause 8: The formulation of any of clauses 1-7, wherein the therapeutic composition is tenofovir disoproxil.

Clause 9: The formulation of any of clauses 1-8, wherein the composition comprises from about 0.1 mg/ml to about 20 mg/ml, about 1.0 mg/ml to about 10 mg/ml, or about 5.28 mg/ml of the therapeutic composition.

Clause 10: The formulation of any of clauses 1-9, wherein the composition comprises from about 0.1 mg/ml to about 20 mg/ml of the therapeutic composition.

Clause 11: The formulation of any of clauses 1-10, wherein the composition comprises from about 1.0 mg/ml to about 10 mg/ml of the therapeutic composition.

Clause 12: The formulation of any of clauses 1-11, wherein the composition comprises about 5.28 mg/ml of the therapeutic composition.

Clause 13: The formulation of any of clauses 1-12, wherein the griffithsin protein is Griffithinsin and/or Q-Griffithsin.

Clause 14: The formulation of any of clauses 1-13, wherein the composition comprises from about 0.01 mg/ml to about 10 mg/ml, about 0.1 mg/ml to about 1 mg/ml, about 0.1 mg/ml to about 0.5 mg/ml, or about 0.32 mg/ml of the griffithsin protein.

Clause 15: The formulation of any of clauses 1-14, wherein the composition comprises from about 0.01 mg/ml to about 10 mg/ml of the griffithsin protein.

Clause 16: The formulation of any of clauses 1-15, wherein the composition comprises from about 0.1 mg/ml to about 1 mg/ml of the griffithsin protein.

Clause 17: The formulation of any of clauses 1-16, wherein the composition comprises from about 0.1 mg/ml to about 0.5 mg/ml of the griffithsin protein.

Clause 18: The formulation of any of clauses 1-17, wherein the composition comprises about 0.32 mg/ml of the griffithsin protein.

Clause 19: The formulation of any of clauses 1-18, wherein the composition is hypotonic, such as having an osmolality from about 100 mOsm/kg to about 200 mOsm/kg, about 123 mOsm/kg to about 332 mOsm/kg, about 110 mOsm/kg to about 180 mOsm/kg, about 123 mOsm/kg to about 167 mOsm/kg, about 137 mOsm/kg, about 139 mOsm/kg, or about 145 mOsm/kg.

Clause 20: The formulation of any of clauses 1-19, wherein the composition has an osmolality from about 100 mOsm/kg to about 200 mOsm/kg.

Clause 21: The formulation of any of clauses 1-20, wherein the composition has an osmolality from about 110 mOsm/kg to about 180 mOsm/kg.

Clause 22: The formulation of any of clauses 1-21, wherein the composition has an osmolality from about 123 mOsm/kg to about 167 mOsm/kg.

Clause 23: The formulation of any of clauses 1-22, wherein the composition has an osmolality of about 137 mOsm/kg.

Clause 24: The formulation of any of clauses 1-23, wherein the composition has an osmolality of about 139 mOsm/kg.

Clause 25: The formulation of any of clauses 1-24, wherein the composition has an osmolality of about or about 145 mOsm/kg.

Clause 26: The formulation of any of clauses 1-25, wherein the composition has a pH from about 6.0 to about 8.5, about 6.5 to about 8.0, or about 7.0.

Clause 27: The formulation of any of clauses 1-26, wherein the composition has a pH from about 6.0 to about 8.5.

Clause 28: The formulation of any of clauses 1-27, wherein the composition has a pH from about 6.5 to about 8.0.

Clause 29: The formulation of any of clauses 1-28, wherein the composition has a pH of about 7.0.

Clause 30: The formulation of any of clauses 1-29, wherein NaOH, HCl, acetic acid, or citric acid is used to adjust the pH of the composition.

Clause 31: The formulation of any of clauses 1-30, wherein NaOH is used to adjust the pH of the composition.

Clause 32: The formulation of any of clauses 1-31, wherein HCl is used to adjust the pH of the composition.

Clause 33: The formulation of any of clauses 1-32, wherein acetic acid is used to adjust the pH of the composition.

Clause 34: The formulation of any of clauses 1-33, wherein citric acid is used to adjust the pH of the composition.

Clause 35: The formulation of any of clauses 1-34, wherein the excipient is a disaccharide or sugar polyol, for example maltitol, lactose, glucose, sorbitol, sucrose, or trehalose.

Clause 36: The formulation of any of clauses 1-35, wherein the excipient is a disaccharide.

Clause 37: The formulation of any of clauses 1-36, wherein the excipient is a sugar polyol.

Clause 38: The formulation of any of clauses 1-37, wherein the excipient is maltitol.

Clause 39: The formulation of any of clauses 1-38, wherein the excipient is lactose.

Clause 40: The formulation of any of clauses 1-39, wherein the excipient is glucose.

Clause 41: The formulation of any of clauses 1-40, wherein the excipient is sorbitol.

Clause 42: The formulation of any of clauses 1-40, wherein the excipient is sucrose.

Clause 43: The formulation of any of clauses 1-40, wherein the excipient is trehalose.

Clause 44: The formulation of any of clauses 1-43, wherein the buffer is phosphate-buffered saline or 0.9% NaCl saline.

Clause 45: The formulation of any of clauses 1-44, wherein the buffer is phosphate-buffered saline.

Clause 46: The formulation of any of clauses 1-45, wherein the buffer is 0.9% NaCl saline.

Clause 47: The formulation of any of clauses 1-46, wherein the composition is clear and/or colorless.

Clause 48: The formulation of any of clauses 1-47, wherein the composition is clear.

Clause 49: The formulation of any of clauses 1-48, wherein the composition is colorless.

Clause 50: The formulation of any of clauses 1-49, wherein the composition is stable for at least two years.

Clause 51: The formulation of any of clauses 1-50, contained within a rectal delivery device.

Clause 52: The formulation of clause 51, wherein the rectal delivery device is an enema bag.

Clause 53: The formulation of clause 51, wherein the rectal delivery device is an enema bag having an extended tip.

Clause 54: The formulation of clause 51, wherein the rectal delivery device is an enema bottle.

Clause 55: The formulation of clause 51, wherein the rectal delivery device is an enema bottle having an extended tip.

Clause 56: A method of producing the formulation of any of clauses 1-55, comprising diluting the buffer and lowering the osmolality to form a first premix, adding the therapeutic composition to the first premix and mixing until the therapeutic composition is completely dissolved to form a second premix, adjusting the osmolality or the pH of the second premix, adding the griffithsin protein to the second premix under constant mixing to form a mixture, testing the pH or osmolality of the mixture and, if necessary, adjusting the pH to between 6.5-8 or adjusting the osmolality to between 123 mOsm/kg and 167 mOsm/kg, and adding the mixture to a rectal delivery device.

Clause 57: The method of clause 56, wherein the griffithsin protein is GRFT and/or Q-GRFT, and, optionally, the Q-GRFT is provided as a Q-GRFT lyophilized powder prepared by a process comprising dissolving the Q-GRFT lyophilized with a disaccharide or sugar polyol, a for example, maltitol, lactose, glucose, sorbitol, sucrose or trehalose, in an aqueous solvent, followed by drying, lyophilizing, or spray-drying the Q-GRFT-containing mixture.

Clause 58: The method of clause 56 or clause 57, wherein the griffithsin protein is Q-GRFT, and the Q-GRFT is provided as a Q-GRFT lyophilized powder prepared by a process comprising dissolving the Q-GRFT lyophilized with a disaccharide or sugar polyol, for example maltitol, lactose, glucose, sorbitol, sucrose or trehalose, in an aqueous solvent, followed by drying, lyophilizing, or spray-drying the Q-GRFT-containing mixture.

Clause 59: A method of treating or preventing a sexually transmitted infection comprising intrarectally delivering the formulation of any of clauses 1-55 to a patient in a dosage regimen effective to treat or prevent the sexually transmitted infection.

Clause 60: The method of clause 59, wherein the sexually transmitted infection is a human immunodeficiency virus (HIV) and/or a herpes virus.

Clause 61: The method of clause 59 or clause 60, wherein the sexually transmitted infection is a human immunodeficiency virus (HIV).

Clause 62: The method of any of clauses 59-61, wherein the sexually transmitted infection is a herpes virus.

Clause 63: The method of any of clauses 59-62, wherein the HIV is HIV-1 or HIV-2, or wherein the herpes virus is a herpes simplex virus.

Clause 64: The method of any of clauses 59-63, wherein the HIV is HIV-1.

Clause 65: The method of any of clauses 59-64, wherein the HIV is HIV-2.

Clause 66: The method of any of clauses 59-65, wherein the herpes virus is a herpes simplex virus.

Clause 67: A method of providing prophylactic protection from a human immunodeficiency virus (HIV), comprising placing the formulation as described herein intrarectally or orally in a patient.

Clause 68: The method of clause 67, comprising placing the formulation as described herein intrarectally.

Clause 69: The method of clause 67 or clause 68, comprising placing the formulation as described herein orally.

Clause 70: A rectal dosage form, comprising a formulation comprising the composition of any of clauses 1-55 in the rectal delivery device.

Clause 71: The rectal dosage form of clause 70 is configured in a liquid dosage form, a solid dosage form, or a semi-solid dosage form.

Clause 72: The rectal dosage form of clause 71 is configured in the liquid dosage form.

Clause 73: The rectal dosage form of clause 71 or clause 72 is configured in the solid dosage form.

Clause 74: The rectal dosage form of any of clauses 71-73 is configured in the semi-solid dosage form.

Clause 75: The rectal dosage form of any of clauses 71-74, wherein the liquid dosage form is a solution, a suspension, or an emulsion.

Clause 76: The rectal dosage form of any of clauses 71-75, wherein the liquid dosage form is a solution.

Clause 77: The rectal dosage form of any of clauses 71-76, wherein the liquid dosage form is a suspension.

Clause 78: The rectal dosage form of any of clauses 71-77, wherein the liquid dosage form is an emulsion.

Clause 79: The rectal dosage form of any of clauses 71-78, wherein the solid dosage form is a suppository, a capsule, a tablet, and/or a powder form.

Clause 80: The rectal dosage form of any of clauses 71-79, wherein the solid dosage form is a suppository.

Clause 81: The rectal dosage form of any of clauses 71-80, wherein the solid dosage form is a capsule.

Clause 82: The rectal dosage form of any of clauses 71-81, wherein the solid dosage form is a tablet.

Clause 83: The rectal dosage form of any of clauses 71-82, wherein the solid dosage form is a powder.

Clause 84: The rectal dosage form of any of clauses 71-83, wherein the semi-solid dosage form is a gel, a foam, and/or a cream.

Clause 85: The rectal dosage form of any of clauses 71-84, wherein the semi-solid dosage form is a gel.

Clause 86: The rectal dosage form of any of clauses 71-85, wherein the semi-solid dosage form is a foam.

Clause 87: The rectal dosage form of any of clauses 71-85, wherein the semi-solid dosage form is a cream.

Clause 88: A method of producing a stable Q-GRFT lyophilized powder comprising dissolving the Q-GRFT lyophilized with a disaccharide or sugar polyol, for example, maltitol, lactose, glucose, sorbitol, sucrose or trehalose, in an aqueous solvent, followed by drying, lyophilizing, or spray-drying the Q-GRFT-containing mixture.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A is a graph of pH stability testing for a PBS-base combination enema formulation as measured under three conditions: 4° C./60% RH for 24 months, 25° C./60% RH for 24 months, 40° C./75% RH for 6 months.

FIG. 1B is a graph of pH stability testing for a Saline-base combination enema formulation as measured under three conditions: 4° C./60% RH for 24 months, 25° C./60% RH for 24 months, 40° C./75% RH for 6 months.

FIG. 2A is a graph of osmolality stability testing for a PBS-base combination enema formulation as measured under three conditions: 4° C./60% RH for 24 months, 25° C./60% RH for 24 months, 40° C./75% RH for 6 months. For all time points, N=3.

FIG. 2B is a graph of osmolality stability testing for a Saline-base combination enema formulation as measured under three conditions: 4° C./60% RH for 24 months, 25° C./60% RH for 24 months, 40° C./75% RH for 6 months. For all time points, N=3.

FIG. 3A is a graph of TFV content stability for a PBS-base combination enema formulation as measured using UPLC, relative content (%, compared to the label claim), and monitored under three conditions: 4° C./60% RH for 24 months, 25° C./60% RH for 24 months, 40° C./75% RH for 6 months. At 0 months, N=6; for all other time points, N=3.

FIG. 3B is a graph of TFV content stability for a Saline-base combination enema formulation as measured using UPLC, relative content (%, compared to the label claim), and monitored under three conditions: 4° C./60% RH for 24 months, 25° C./60% RH for 24 months, 40° C./75% RH for 6 months. At 0 months, N=6; for all other time points, N=3.

FIG. 4A is a graph of Q-GRFT content stability for a PBS-base combination enema formulation as measured using HPLC, relative content (%, compared to the label claim), and monitored under three conditions: 4° C./60% RH for 24 months, 25° C./60% RH for 24 months, 40° C./75% RH for 6 months. At 0 months, N=6; for all other time points, N=3.

FIG. 4B is a graph of Q-GRFT content stability for a Saline-base combination enema formulation as measured using HPLC, relative content (%, compared to the label claim), and monitored under three conditions: 4° C./60% RH for 24 months, 25° C./60% RH for 24 months, 40° C./75% RH for 6 months. At 0 months, N=6; for all other time points, N=3.

FIG. 5A is a graph of in vitro Caco-2 cell toxicity of the excipient Na2HPO4 in the enema formulations, with red lines indicating the highest concentrations used in the enema formulations. N=8 for all groups.

FIG. 5B is a graph of in vitro Caco-2 cell toxicity of the excipient KH₂PO₄ in the enema formulations, with red lines indicating the highest concentrations used in the enema formulations. N=8 for all groups.

FIG. 5C is a graph of in vitro Caco-2 cell toxicity of the excipient KCl in the enema formulations, with red lines indicating the highest concentrations used in the enema formulations. N=8 for all groups.

FIG. 6 is a graph of in vitro toxicity of two combination enema formulations using the Caco-2 monolayer cell model. Cell viability compared to the untreated group (100%) using the 3-(4,5-dimethylthiazol-2-yl)-2,5-diphenyl-2H-tetrazolium bromide (MTT) method. N=3.

FIG. 7 is a graph of in vitro toxicity of two combination enema formulations using the Caco-2 monolayer cell model. TEER values after the treatments. The black dot line indicates the lower threshold. N=3 for the untreated and the formaldehyde groups. N=9 for both combo enema groups.

FIG. 8 is a multicolor photograph of in vitro toxicity of two combination enema formulations using the Caco-2 monolayer cell model. Confocal images were taken by a Nikon AIR confocal microscope. The tight junction protein (ZO-1) was stained in green and the nuclei in blue. Scales (20 μm) are shown in the pictures.

FIG. 9 is a multicolor graph of cumulative [¹⁴C]-Mannitol content over time in multiple enema formulations using the Caco-2 monolayer cell model. [¹⁴C]-Mannitol was used as the paracellular marker and added in all the groups. Samples from the receptor sides were collected over two hours and measured for [¹⁴C]. N=3 for all groups.

FIG. 10 is a graph of apparent permeability (Papp) TFV in four hypotonic enema formulations using human colon tissues in the Ussing Chamber. Black dots indicate individual values, while bars indicate mean values with standard deviations. Data in each group contains replicate N=8-20, from 3-7 tissue donors. One-way ANOVA was performed using Prism 9. No significance was found between any two groups.

FIG. 11 is a graph of bioactivity of gp120 binding efficacy as measured via ELISA. N=3 for all groups. No significance was observed for the EC50 across groups.

FIG. 12 is a graph of Q-GRFT content detected via HPLC in sugar/sugar polyol cryoprotectants after three months and after storing the lyophilized powder under an accelerated condition (40° C./75% RH). N=4. *p<0.05.

FIG. 13 is a graph of Q-GRFT content detected via HPLC in maltilol cryoprotectants. The Maltitol ratio was defined by the molar ratio of maltitol to Q-GRFT monomer. Q-GRFT content was detected at three months by HPLC after storing the lyophilized powder under an accelerated condition (40° C./75% RH). N=4. *p<0.05; **p<0.01; ****p<0.0001.

FIG. 14 is a graph of Q-GRFT content detected via HPLC in maltilol cryoprotectants. Maltitol ratio was defined by the molar ratio of maltitol to Q-GRFT monomer. Q-GRFT content was detected at three months by HPLC after storing the lyophilized powder under a normal condition (25° C./60% RH). N=4. A dashed line indicates the 100% label claim.

FIG. 15A is a graph of Q-GRFT content detected via HPLC after storing the lyophilized powder under a normal condition (25° C./60% RH). N=4. Dash lines indicate the 90% (lower limit), 100% (target), and 110% (upper limit) of the label claim.

FIG. 15B is a multicolor graph depicting stability of the enema formulations as detected via SEC chromatography. LMP group (shown in blue) and the Q-GRFT reference group (shown in magenta) were compared. Q-GRFT elutes at the same retention time for both groups. No aggregation peaks were detected.

FIG. 16 is a multicolor graph of crystallizations detected in LMP (green line), Q-GRFT only lyophilized powder (red line), maltilol only lyophilized powder (magenta line), and maltilol only un-lyophilized powder (blue line), as measured via DSC chromatography in relative energy over temperature. Negative peaks indicate endothermic peaks.

FIG. 17 is a multicolor graph of crystallization patterns detected in Q-GRFT only lyophilized powder (blue line), LMP (black line), PBS lyophilized (green line), and maltilol lyophilized (red line) as measured via XRD in intensity over 2θ.

FIG. 18 is a graph of bioactivity of Q-GRFT LMP determined by gp120 binding efficacy, as measured via ELISA. N=3.

FIG. 19 is a graph of TFV content in multiple formulations over time as detected via UPLC. TFV was lyophilized with two base formulations (Saline-base and PBS-base). TFV content was detected at different time points by UPLC after storing the lyophilized powder under an accelerated condition (40° C./75% RH). Dash lines indicate the lower (90%) and upper (110%) limits of the label claim. N=3.

FIG. 20 is a graph of Q-GRFT content detected in individual or combined reconstituted enema. N=3. No significance was detected among the groups. Dash lines indicate the 90% (lower limit) and 110% (upper limit) of the label claim.

FIG. 21 is a graph of TFV content detected in individual or combined reconstituted enema. N=3. No significance was detected among the groups. Dash lines indicate the 90% (lower limit) and 110% (upper limit) of the label claim.

FIG. 22 is a table of apparent permeability (Papp) values of TFV in four hypotonic enema formulations tested in human colon tissue samples and measured with an Ussing chamber.

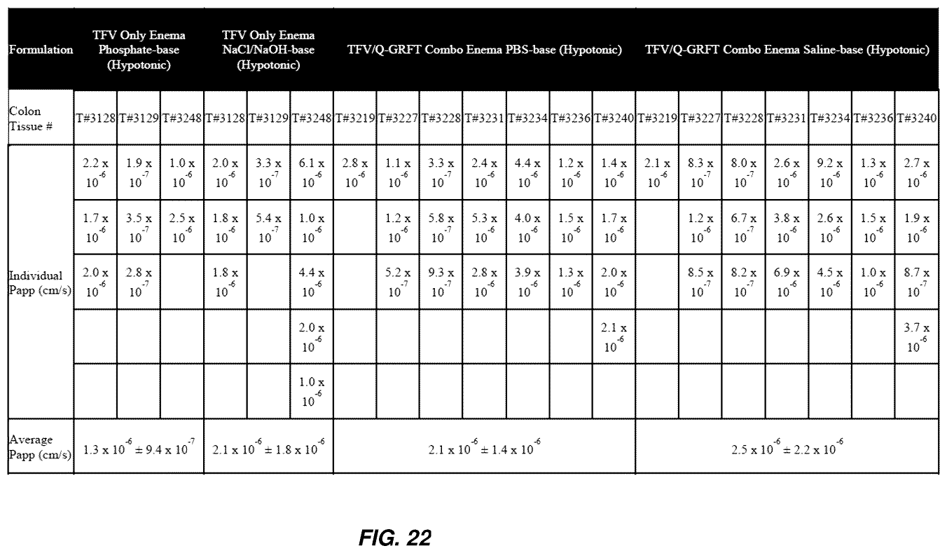

FIG. 23 is a multicolor graph of TEER values in human colon tissue samples exposed to combination enema formulations and measured with an Ussing Chamber. Six samples of human colon tissue were obtained from the same donor patients. TEER values were monitored through the experiments over two hours, and are reported as % to the Time 0 TEER value. PBS- and Saline-base combo formulations were performed in triplicates, numbered as 1, 2, and 3 respectively, and shown in different colored lines on the graph.

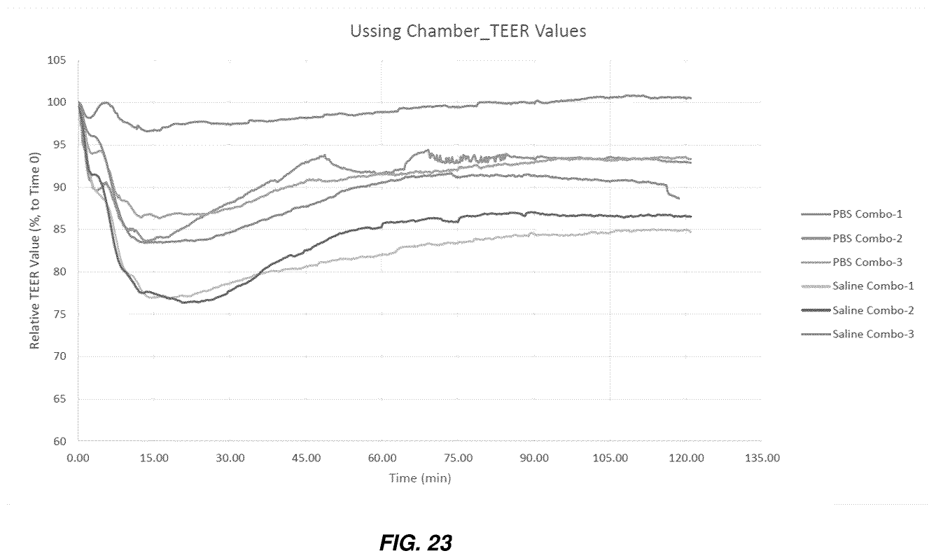

FIG. 24 is a three-panel series of multicolor photographs displaying epithelial structures in human colon tissue samples stained via H&E staining. The left panel is a tissue sample stained before a two-hour treatment. The center panel is a tissue sample stained after treatment with PBS-base combo formulation. The right panel is a tissue sample stained after treatment with Saline-base combo enema.

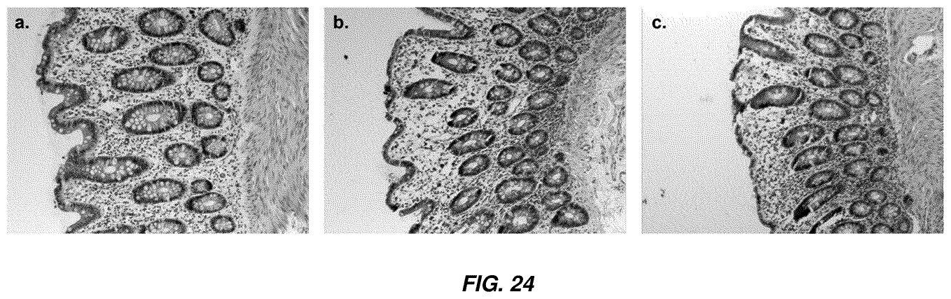

FIG. 25 is a graph of the results of a short-term (10-day) stability study of a Q-GRFT-only (in PBS) lyophilized powder formulation. Samples (N=3) were stored in real-time conditions (25° C./60% RH). Q-GRFT content was detected at different time points via HPLC, and significant differences (p<0.05) from Day 0 were found for Days 3, 4, 7, 8, and 10.

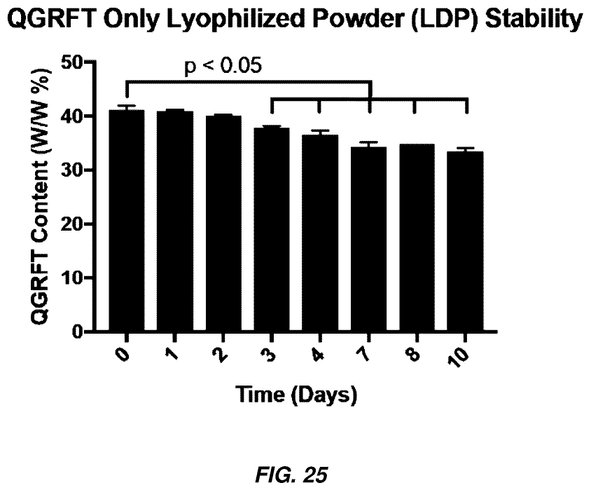

FIG. 26 is a graph of the results of a short-term (1-month) stability study of a Q-GRFT-only (in PBS) lyophilized powder formulation. Samples were stored in real-time conditions (25° C./60% RH). Elution time and aggregation were measured via SEC chromatography in the experimental formulation (bottom) and the Q-GRFT reference group (top) on Day 0 (left) and Day 30 (right). Q-GRFT monomer peaks elute at the same retention time for all four chromatographs. Aggregation was detected for the Q-GRFT only lyophilized powder group on Day 30 (arrow).

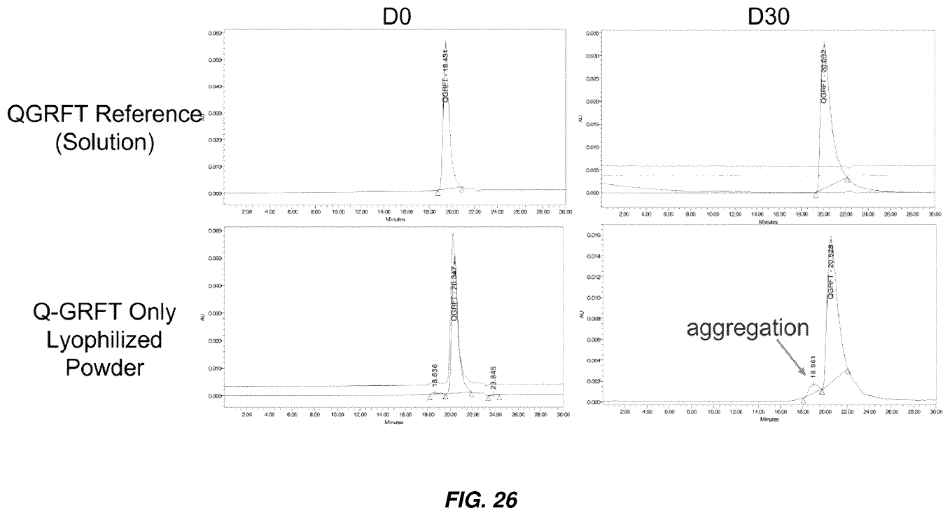

FIG. 27 is a graph of crystallization of individual excipients in PBS (non-lyophilized) measured via DSC chromatography. Relative energy (y-axis) is plotted against temperature (x-axis), with negative peaks indicating endothermic peaks, and integration of the peaks shown in the figure. Tested excipients include KCl (top), NaCl (second from the top), Na₂HPO₄ (third from the top), and Na₂HPO₄ in a different quantity (bottom).

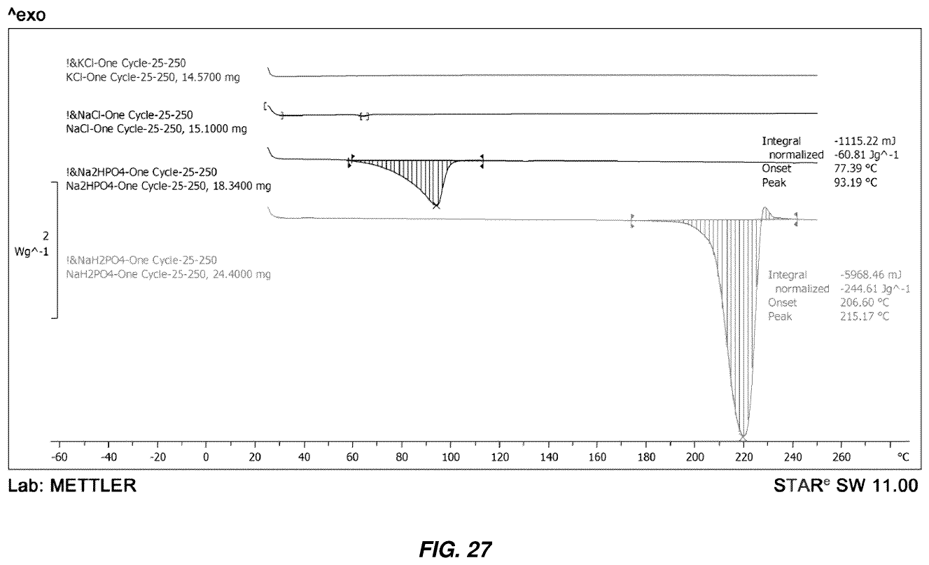

FIG. 28 is a graph of crystallization of a combination of excipients in PBS, the excipients being KCl, NaCl, Na₂HPO₄, and Na₂HPO₄. Relative energy (y-axis) is plotted against temperature (x-axis), with negative peaks indicating endothermic peaks, and integration of the peaks shown in the figure.

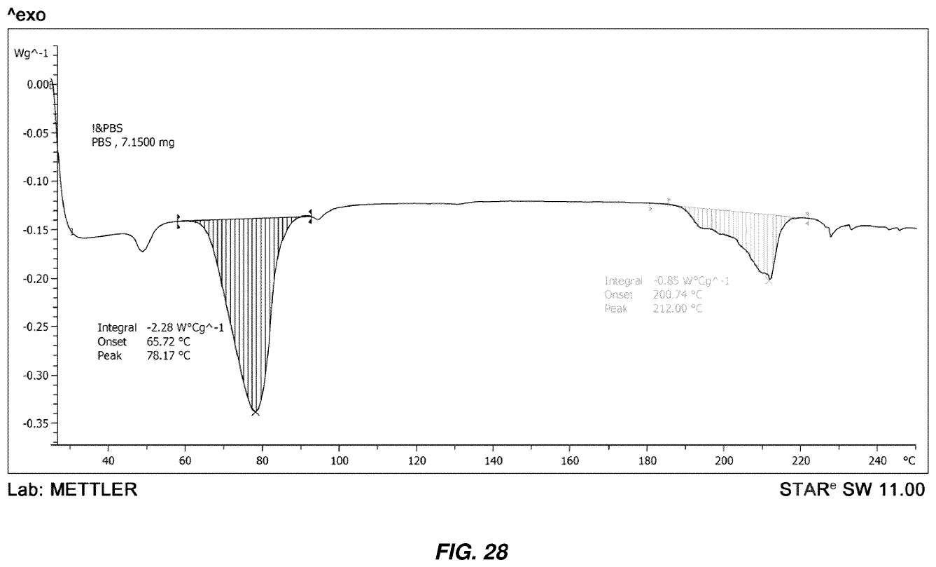

FIG. 29 is a graph of XRD patterns for the PBS lyophilized powder, plotted using Prism 9. Intensity (y-axis) was plotted against 2θ (x-axis), and signature peaks were identified based on data from the literature.

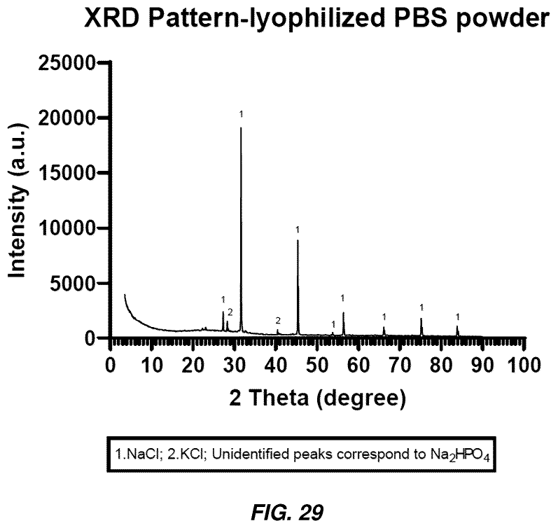

FIG. 30A is a graph of the results of a stability study of TFV lyophilized (with maltitol) powder formulation. TFV was lyophilized with maltitol in a Saline-base formulation. TFV content was detected at different time points via UPLC after storing the lyophilized powder under an accelerated condition (40° C./75% RH). N=3. Dash lines indicate the lower (90%) and upper (110%) limits of the label claim.

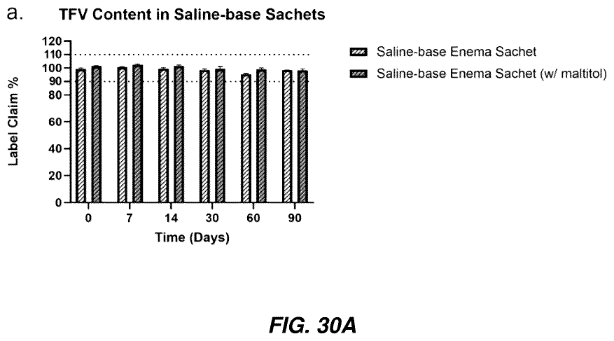

FIG. 30B is a graph of the results of a stability study of TFV lyophilized (with maltitol) powder formulation. TFV was lyophilized with maltitol in a PBS-base formulation. TFV content was detected at different time points via UPLC after storing the lyophilized powder under an accelerated condition (40° C./75% RH). N=3. Dash lines indicate the lower (90%) and upper (110%) limits of the label claim.

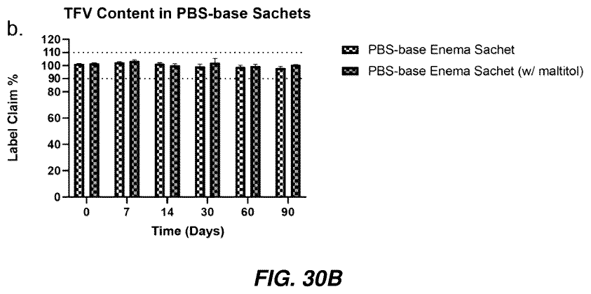

FIG. 31 is a graph of pH values in individual or combined reconstituted enema (target pH range is 6.5-8). N=3.

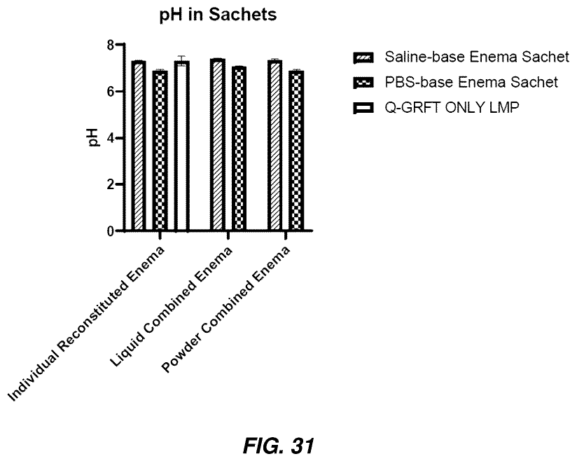

FIG. 32 is a graph of osmolality values in individual or combined reconstituted enema. N=3. Dash lines show the 85% (lower limit) and 115% (upper limit) of the target osmolality. Target osmolality for combo enema is 145 mOsm/kg. LMP has an osmolality close to 0 due to the lack of buffering agents.

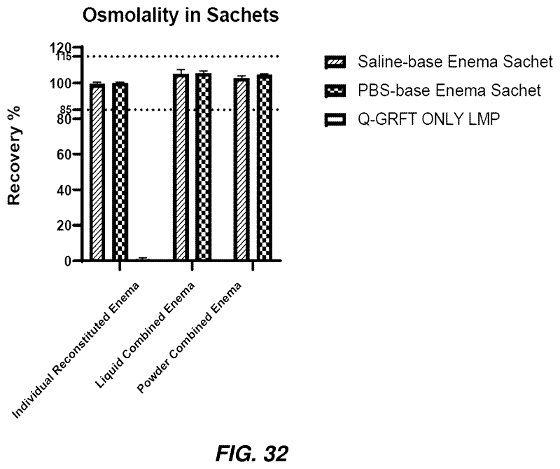

FIG. 33 is a graph of the results of a long-term stability study of LMP. Q-GRFT content was detected at different time points by HPLC after storing the lyophilized powder under a real-time condition (25° C./60% RH). For Time 0 and 12 months, N=4. For Time 24 months, N=6. Dash lines indicate the 90% (lower limit), 100% (target), and 110% (upper limit) of the label claim. NS=No significance.

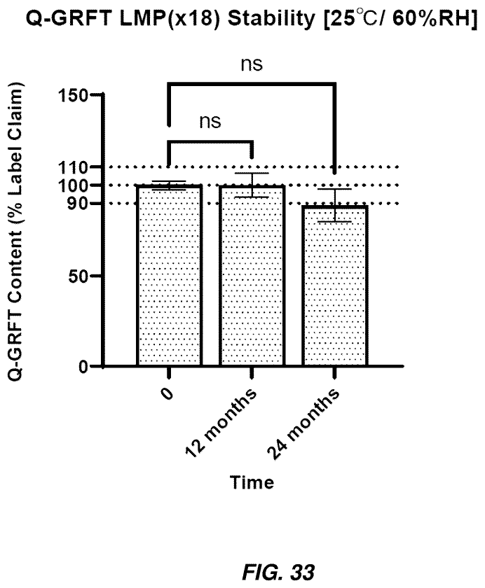

FIG. 34 is a graph of a long-term stability study of LMP. Elution and aggregation were measured via SEC chromatography. Samples were stored under a real-time condition (25° C./60% RH) and tested on the last time point (24 months). LMP group (shown in black) and the Q-GRFT reference group (shown in gray) were compared. Q-GRFT elutes at the same retention time for both groups. An aggregation peak was detected as indicated in the figure.

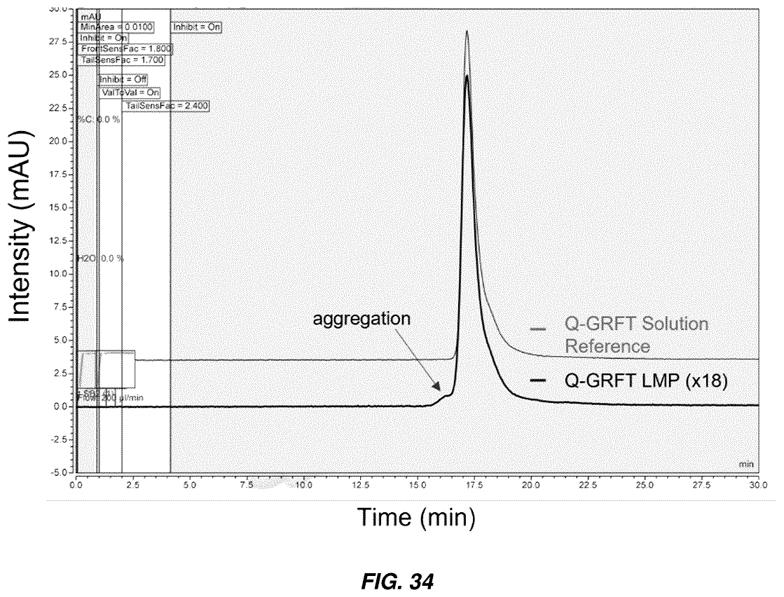

### DESCRIPTION OF THE INVENTION

The following description is merely exemplary in nature and is in no way intended to limit the invention, its application, or uses. While the description is designed to permit one of ordinary skill in the art to make and use the invention, and specific examples are provided to that end, they should in no way be considered limiting. It will be apparent to one of ordinary skill in the art that various modifications to the following will fall within the scope of the appended claims. The present invention should not be considered limited to the presently disclosed aspects, whether provided in the examples or elsewhere herein.

The use of numerical values in the various ranges specified in this application, unless expressly indicated otherwise, are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of these ranges is intended as a continuous range including every value between the minimum and maximum values. For definitions provided herein, those definitions refer to word forms, cognates and grammatical variants of those words or phrases. As used herein “a” and “an” refer to one or more. Patent publications cited below are hereby incorporated herein by reference in their entirety to the extent of their technical disclosure and consistency with the present specification.

As used herein, the terms “comprising,” “comprise,” “comprised,” and variations thereof, are open ended and do not exclude the presence of other elements not identified. In contrast, the term “consisting of” and variations thereof is intended to be closed and excludes additional elements in anything but trace amounts.

As used herein, the term “patient” or “subject” refers to members of the animal kingdom including but not limited to human beings and “mammal” refers to all mammals, including, but not limited to human beings.

As used herein, the “treatment” or “treating” of a sexually transmitted infection (STI) means administration to a patient by any suitable dosage regimen, procedure, and/or administration route of a composition, device, or structure with the object of achieving a desirable clinical/medical end-point, including but not limited to, for a STI, reducing or preventing further development of the STI, e.g., as determined below. An amount of any reagent or therapeutic agent, administered by any suitable route, effective to treat a patient is an amount capable of preventing, reducing, and/or eliminating the STI, and/or reducing the severity of one or more symptoms of the STI, for example, discharge from the vagina, penis, or anus, pain when urinating, lumps or skin growths around the genitals or anus, a rash, vaginal bleeding, pruritus of the genitals or anus, blisters or sores around the genitals or anus or throat, or warts around the genitals or anus. The therapeutically-effective amount of each therapeutic may range from about 1 pg per dose to about 10 g per dose, including any amount and subrange therebetween, such as, without limitation, about 1 ng, about 1 μg, about 1 mg, about 10 mg, about 100 mg, or about 1 g per dose. The therapeutic agent may be administered by any effective route, and, for example, as a single dose or bolus, at regular or irregular intervals, in amounts and intervals as dictated by any clinical parameter of a patient, or continuously.

Active ingredients, such as an antiretroviral composition or a griffithsin protein, may be compounded, formulated, or otherwise manufactured into a suitable composition for use, such as a pharmaceutical dosage form, a rectal dosage form, or drug product in which the compound is an active ingredient. Compositions may comprise a pharmaceutically acceptable carrier, or excipient. An excipient is an inactive substance used as a carrier for the active ingredients of a medication. Although “inactive,” excipients may facilitate and aid in increasing the delivery or bioavailability of an active ingredient in a drug product. Non-limiting examples of useful excipients include: antiadherents, stabilizers, binders, rheology modifiers, coatings, disintegrants, emulsifiers, oils, buffers, salts, acids, bases, fillers, diluents, solvents, flavors, colorants, glidants, lubricants, preservatives, antioxidants, sorbents, vitamins, sweeteners, etc., as are available in the pharmaceutical/compounding arts. Additional non-limiting examples of useful excipients include disaccharides or sugar polyols (e.g., lactose, glucose, sorbitol, and maltitol).

Useful dosage forms include: intrarectal, intravenous, intramuscular, intraocular, or intraperitoneal solutions, oral tablets or liquids, topical ointments or creams, and transdermal devices (e.g., patches). In one non-limiting embodiment, the compound is a sterile solution comprising the active ingredient (drug, or compound), and a solvent, such as water, saline, lactated Ringer's solution, or phosphate-buffered saline (PBS). In non-limiting embodiments, the compound is non-sterile. Additional excipients, such a disaccharides or sugar polyols, polyethylene glycol, emulsifiers, salts, and buffers may be included in the solution.

Suitable dosage forms may include single-dose, or multiple-dose, sachet pouches, powder pouches, vials or other containers, such as an enema bag, an enema bottle, an enema bottle having an extended tip, medical syringes or droppers, containing a composition comprising an active ingredient useful for treatment of an infection as described herein. Additional dosage forms may include a rectal dosage form configured in a liquid dosage form, a solid dosage form, or a semi-solid dosage form. In some embodiments, the liquid dosage form is a solution, a suspension, or an emulsion. In some embodiments, the solid dosage form is a suppository, a capsule, or a tablet, or a powder form. In some embodiments, the semi-solid dosage form is a gel, a foam, or a cream. In one embodiment or example, the dosage form is a liquid rectal dosage form, such as a liquid comprising tenofovir and griffithsin active ingredients, for example as disclosed herein, contained within an enema bag, an enema bottle, or an enema bottle having an extended tip.

Pharmaceutical formulations adapted for administration include aqueous and non-aqueous sterile and non-sterile solutions which may contain, for example and without limitation, anti-oxidants, buffers, bacteriostats, lipids, liposomes, lipid nanoparticles, emulsifiers, suspending agents, and rheology modifiers. The formulations may be presented in unit-dose or multi-dose containers, for example, sealed ampoules and vials, and may be stored in a freeze-dried (lyophilized) condition requiring only the addition of the sterile liquid carrier, for example, water for injections, immediately prior to use. Extemporaneous solutions and suspensions may be prepared from sterile powders, granules, and tablets.

Therapeutic compositions typically must be sterile, though the lyophilized powders and reconstituted enema solutions described herein need not be so. Therapeutic compositions typically must be stable under the conditions of manufacture and storage. For example, sterile injectable solutions can be prepared by incorporating the active agent in the required amount in an appropriate solvent with one or a combination of ingredients enumerated herein, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, typical methods of preparation are vacuum drying, freeze-drying, or spray-drying that yields a powder of the active ingredient plus any additional desired ingredient from a previously sterile-filtered solution thereof. The proper fluidity of a solution can be maintained, for example, by the use of an emulsifier such as lecithin, by the maintenance of the required particle size in the case of dispersion, and by the use of surfactants and/or rheology modifiers. Prolonged absorption of injectable compositions can be brought about by including in the composition an agent that delays absorption, for example, monostearate salts and gelatin.

A “therapeutically effective amount” refers to an amount of a drug product or active agent effective, at dosages and for periods of time necessary, to achieve the desired therapeutic result. An “amount effective” for treatment of a condition is an amount of an active agent or dosage form, such as a single dose or multiple doses, effective to achieve a determinable end-point. The “amount effective” is preferably safe-at least to the extent the benefits of treatment outweigh the detriments, and/or the detriments are acceptable to one of ordinary skill and/or to an appropriate regulatory agency, such as the U.S. Food and Drug Administration. A therapeutically effective amount of an active agent may vary according to factors such as the disease state, age, sex, and weight of the individual, and the ability of the active agent to elicit a desired response in the individual. A “prophylactically effective amount” refers to an amount effective, at dosages and for periods of time necessary, to achieve a desired prophylactic result. Typically, since a prophylactic dose is used in subjects prior to or at an earlier stage of disease, the prophylactically effective amount may be less than the therapeutically effective amount.

Dosage regimens may be adjusted to provide an optimum desired response (e.g., a therapeutic or prophylactic response). For example, a single dose or bolus may be administered, several divided doses may be administered over time, or the composition may be administered continuously or in a pulsed fashion with doses or partial doses being administered at regular intervals, for example, every 10, 15, 20, 30, 45, 60, 90, or 120 minutes, every 2 through 12 hours daily, or every other day, etc. The composition may be proportionally reduced or increased as indicated by the exigencies of the therapeutic situation. In some instances, it may be especially advantageous to formulate compositions in dosage unit form for ease of administration and uniformity of dosage. The specification for the dosage unit forms are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

Provided herein are stable pharmaceutical compositions (e.g., drug products) in the form of a liquid formulation, such as a hypotonic liquid formulation, of various therapeutic compositions that may be advantageously delivered using a rectal delivery device (e.g., an enema bag or enema bottle). In some embodiments, the formulation may comprise two or more therapeutic compositions useful in the treatment or prevention of a sexually transmitted infection (STI). The formulations described herein are the first enema solutions designed for delivering synergistic entities for STI prevention or treatment. When compared to other single-entity enema pre-exposure prophylaxis (PrEP) options, the combination enema has the benefit to offer superior protection. The formulations described herein may utilize a hypotonic solution which has been shown to enhance drug penetration of any of the therapeutic compositions described herein, such as tenofovir.

In some embodiments, a formulation, optionally contained in a rectal delivery device is provided. The formulation may comprise a therapeutic composition and a griffithsin protein. The formulation is optionally hypertonic, hypotonic, or isotonic. In some embodiments, the therapeutic composition and the griffithsin protein may be provided in a composition comprising an excipient and a buffer.

Tonicity is a measure of the effective osmotic pressure gradient, for example, the water potential of two solutions separated by a semipermeable cell membrane. Tonicity depends on the relative concentration of selectively membrane permeable solutes across a cell membrane which determine the direction and extent of osmotic flux. It is commonly used when describing the swelling versus shrinking response of cells immersed in an external solution. A hypotonic solution has a lower concentration of solutes than another solution. In biology, a solution outside of a cell is called hypotonic if it has a lower concentration of solutes relative to the cytosol. Due to osmotic pressure, water diffuses into the cell, and the cell often appears turgid, or bloated. For cells without a cell wall such as animal cells, if the gradient is large enough, the uptake of excess water can produce enough pressure to induce cytolysis, or rupturing of the cell. A hypertonic solution has a greater concentration of solutes than another solution. In biology, the tonicity of a solution usually refers to its solute concentration relative to that of another solution on the opposite side of a cell membrane. A solution outside of a cell is called hypertonic if it has a greater concentration of solutes than the cytosol inside the cell. When a cell is immersed in a hypertonic solution, osmotic pressure tends to force water to flow out of the cell in order to balance the concentrations of the solutes on either side of the cell membrane. The cytosol is conversely categorized as hypotonic, opposite of the outer solution.

In some embodiments, the formulation is hypotonic and has an osmolality from about 20 mOsm/kg to about 280 mOsm/kg, from about 123 mOsm/kg to about 332 mOsm/kg, from about 50 mOsm/kg to about 250 mOsm/kg, from about 75 mOsm/kg to about 225 mOsm/kg, from about 100 mOsm/kg to about 200 mOsm/kg, about 110 mOsm/kg to about 180 mOsm/kg, about 123 mOsm/kg to about 167 mOsm/kg, about 137 mOsm/kg, about 139 mOsm/kg, or about 145 mOsm/kg, all values and subranges therebetween for each of the foregoing included.

As used herein, a rectal delivery device is a device used to deliver the formulations or compositions described herein to the rectum. Traditional rectal delivery devices have been used for localized treatments including delivery of laxatives, treatment of hemorrhoids, and for delivery of antipyretics. Rectal delivery devices include, but are not limited to, an enema bag or an enema bottle, e.g., having an extended tip.

In some embodiments, the therapeutic compositions/active ingredients of the formulation useful in the rectal delivery device described herein can include, but not limited to, antiretroviral compositions (e.g., nucleoside reverse transcriptase inhibitors, nonnucleoside reverse transcriptase inhibitors, protease inhibitors, fusion inhibitors, entry inhibitors, and integrase strand transfer inhibitors, such as, for example and without limitation, efavirenz, emtricitabine, rilpivirine, atazanavir sulfate, darunavir ethanolate, elvitegravir, lamivudine, zidovudine, abacavir, zalcitabine, dideoxycytidine, azidothymidine, didanosine, dideoxyinosine, stavudine, rilpivirine, etravirine, delvaridine, nevirapine, amprenavir, tipranavir, inidinavir, saquinavir, lopinavir, ritonavir, fosamprenavir, ritonavir, darunavir, atazanavir, nelfinavir, enfuvirtide, raltegravir, dolutegravir, elvitegravir, maraviroc, DS003, tenofovir (TFV), TFV alafenamide, TFV disoproxil fumarate, and dapivirine), antiviral compositions (e.g., 4′-Ethynyl-2-fluoro-2′-deoxyadenosine (EFdA), nucleoside analogs, such as: acyclovir (2-amino-9-(2-hydroxyethoxymethyl)-3H-purin-6-one), penciclovir (2-amino-9-[4-hydroxy-3-(hydroxymethyl)butyl]-3H-purin-6-one), foscarnet (phosphonoformic acid), cidofovir ([(2S)-1-(4-amino-2-oxopyrimidin-1-yl)-3-hydroxypropan-2-yl]oxymethylphosphonic acid), adefovir (2-(6-aminopurin-9-yl)ethoxymethylphosphonic acid), and pharmaceutically-acceptable ester prodrugs thereof, such as valaciclovir (valine aciclovir ester, 2-[(2-amino-6-oxo-3H-purin-9-yl)methoxy]ethyl (2S)-2-amino-3-methylbutanoate) or famciclovir ([2-(acetyloxymethyl)-4-(2-aminopurin-9-yl)butyl] acetate)), antibiotic/antiprotozoal compositions (e.g., GRFT, CSIC, metronidazole), antifungal compositions (e.g., clotrimazole), hormones or hormonal compositions (e.g., levonorgestrel, etonogrestrel, desogrestrel, dienogest), and hormonal receptor modulators (e.g., ulipristal acetate). In addition, other compounds such as RANTES derivatives and retrocyclin (e.g., RC-101) can be included. Compositions that affect metabolism of another compositions, such as antiretroviral compositions, such as cobicistat (sold under the trade name Tyboost®) can also be included. For example, a composition can include atazanavir and cobicistat (sold under the trade name Evotaz®).

In some embodiments, the therapeutic composition is an antiretroviral composition. In some embodiments, the therapeutic composition is a nucleoside reverse transcriptase inhibitor or a nonnucleoside reverse transcriptase inhibitor. In some embodiments, the therapeutic composition comprises tenofovir or a pharmaceutically acceptable salt thereof. In some embodiments, the therapeutic composition is tenofovir, tenofovir alafenamide, or tenofovir disoproxil. In non-limiting embodiments, a therapeutically-effective dose includes about 500 mg to about 700 mg, optionally about 600 mg to about 700 mg, optionally about 650 mg to about 700 mg, optionally about 650 mg, optionally about 660 mg, of a therapeutic composition (e.g., tenofovir), which may be reconstituted in a volume of about 125 ml of reconstituting solution (e.g., in some non-limiting embodiments, about 1.76 mg/ml to about 10 mg/ml, optionally about 1.76 mg/ml to about 5.28 mg/ml, optionally 5.28 mg/ml in a final reconstituted enema solution, all values and subranges therebetween for all of the foregoing included). In some embodiments, the active ingredient of the therapeutic composition is tenofovir, tenofovir alafenamide, or tenofovir disoproxil, and is present in an amount from about 0.1 mg/ml to about 20 mg/ml, about 1.0 mg/ml to about 10 mg/ml, or about 5.28 mg/ml.

Those of skill in the art will appreciate that the aforementioned examples are non-limiting, and that any therapeutic composition, including antiviral, antiretroviral, antibacterial, antiprotozoal, antifungal, or hormone-based therapeutics, can advantageously be included in rectal delivery devices described herein.

The formulation may be, for example, a stable pharmaceutical composition in the form of a hypertonic, hypotonic, or isotonic formulation for the delivery of TFV (including TFV pharmaceutical salt forms). TFV may be provided as 9-[9(R)-2-(phosphonomethoxy)propyl]adenine (PMPA), tenofovir disoproxil, or as tenofovir alafenamide. The salt forms, for example fumarate salt forms, of either composition can be included in compositions as described herein. For ease of reference, references herein will be made to “tenofovir” or TFV, with the understanding that the term can refer to PMPA, tenofovir disoproxil, or tenofovir alafenamide, as well as salts thereof.

In non-limiting aspects, the formulation is a stable pharmaceutical composition in the form of a formulation, optionally a hypotonic formulation, for the delivery of TFV in combination with another pharmaceutically active agent, for example, a griffithsin protein.

Griffithsin (GRFT) is a protein that was originally isolated from red algae. It binds the terminal mannose residues of N-linked glycans found on the surface of human immunodeficiency virus type 1 (HIV-1), HIV-2, and other enveloped viruses, including hepatitis C virus (HCV), severe acute respiratory syndrome coronavirus (SARS-CoV), various avian CoV subtypes, BCoV, IBV, MHV, PCoV, HCoV and mutants, JEV, SIV, and SHIV. Its activity has also been demonstrated in Nipah, Ebola virus, Herpes, Influenza, and RSV. An engineered form of GRFT, termed Q-GRFT, has increased stability against oxidation and displays similar activity (see, U.S. Pat. No. 10,501,507 B2, the contents of which are herein incorporated by reference). This increased stability is relevant to the design of a marketable pharmaceutical product.

In some embodiments, the griffithsin (GRFT) protein is Q-griffithsin (Q-GRFT) or a combination of GRFT AND Q-GRFT. In some embodiments, the griffithsin protein or Q-griffithsin is in an amount from about 0.01 mg/ml to about 10 mg/ml, about 0.1 mg/ml to about 1 mg/ml, about 0.1 mg/ml to about 0.5 mg/ml, or about 0.32 mg/ml. In non-limiting embodiments, a therapeutically-effective dose of GRFT and/or Q-GRFT includes about 10 mg to about 100 mg, optionally about 20 mg to about 50 mg, optionally about 40 mg, which may be reconstituted in a volume of about 125 ml of reconstituting solution (e.g., in some non-limiting embodiments, about 0.32 mg/ml in a final reconstituted enema solution, all values and subranges therebetween for the foregoing included).

The formulation described herein may include both of TFV and Q-GRFT, and may be hypotonic. The formulation as described herein is particularly suitable for intrarectal administration, and for preexposure prophylaxis (PrEP) to reduce, prevent, or treat a sexually transmitted infection, for example, HIV or herpes.

Also disclosed herein are methods of manufacturing or producing a formulation for rectal delivery optionally in a rectal delivery device. Generally, stock buffer (e.g., 10×-PBS or Saline) is diluted with Milli-Q water to a solution with lower osmolality than desired. TFV dry powder is weighed and added into the solution under constant stirring (e.g., 250-400 rpm) to form a dispersion. Using a pH meter to monitor pH, pH can adjusted using an 18% NaOH solution to the desired pH (e.g., a pH from about 6.0 to about 8.5, from about 6.5 to about 8.0, or about 7.0). Other pH adjusters may include NaOH, HCl, acetic acid, or citric acid. The TFV can be fully dissolved using stirring after pH adjustment. Finally, Q-GRFT stock solution (in 1× PBS) is measured and added into the solution to make the final product. Both PBS- and saline-based formulations may utilize the same procedure.

Also disclosed herein are methods of manufacturing a stable Q-GRFT lyophilized powder. A non-limiting protocol for forming a stable Q-GRFT lyophilized powder includes combining the Q-GRFT stock solution (in 1× PBS) with a disaccharide or sugar polyol, for example maltitol, lactose, glucose, sorbitol, sucrose, or trehalose, and customizing the lyophilization duration with water content monitoring. The final Q-GRFT lyophilized powder can be reconstituted into one or more formulations described herein (e.g., a hypotonic formulation or a hypotonic enema formulation).

Also disclosed herein are methods of treating or preventing a sexually transmitted infection. In some embodiments, the formulation disclosed herein is delivered intrarectally to a patient in a dosage regimen effective to treat or prevent the sexually transmitted infection. In non-limiting embodiments, the dosage form is an enema. In non-limiting embodiments, a kit is provided for this treatment/prevention, the kit including a sachet of a lyophilized composition (including one or more of the therapeutic compositions described herein), and, optionally, a liquid in which the composition may be reconstituted for delivery. In some embodiments, the sexually transmitted infection is HIV or a herpes virus. In some embodiments, the HIV is HIV-1 or HIV-2. In some embodiments, the herpes simplex virus is HSV-1 or HSV-2.

Also disclosed herein are rectal dosage forms. Useful rectal dosage forms for including the formulation described herein are generally described in Hua, S., Physiological and Pharmaceutical Considerations for Rectal Drug Formulations, Front Pharmacol., 2019 Oct. 16, 10:1196, doi: 10.3389/fphar.2019.01196 (PMID: 31680970; PMCID: PMC6805701), the contents of which are hereby incorporated by reference. Useful rectal dosage forms may include a liquid dosage form, for example, a solution, a suspension, or an emulsion. Further useful rectal dosage forms may include a solid dosage form, for example, a suppository, a capsule, or a tablet. In further embodiments, a useful rectal dosage form may include a semi-solid dosage form, for example, a gel, a foam, or a cream.

### Example 1

Materials: Recombinant Q-GRFT drug substance was manufactured by Kentucky Bioprocessing LLC (Owensboro, KY), and supplied by Dr. Kenneth Palmer at the University of Louisville. Tenofovir (TFV) was synthesized by WuXi AppTec (Shanghai, China). PBS 10× molecular biology grade (pH 7.4) was purchased from Mediatech, Inc. (Manassa, VA). Saline buffer (sodium chloride injection, USP, sterile) was purchased from B. Braun Medical Inc. (Irvine, CA). Acetonitrile (ACN), trifluoroacetic acid (TFA), t-butylammonium bisulfate (tBAHS), potassium phosphate dibasic (Na₂HPO₄), hydrochloric acid (HCl), sodium hydroxide (NaOH), Krebs Ringer Bicarbonate Buffer containing Glucose (KRBG), Hank's Balanced Salt Solution (HBSS), and Hematoxylin and Eosin (H&E) stain kit were obtained from Fisher Scientific (Pittsburgh, PA). A MilliQ (Millipore; Milford, MA) water filtration system operating at 18.2 MΩ cm was used for water. PBS 1× was manufactured in-house with PBS 10× stock and MilliQ-water.

To investigate if the vehicle strength impacts the pH and osmolality of formulations, PBS-base was used directly (1× PBS) or diluted (50% PBS) with or without the addition of TFV and Q-GRFT. To study if the vehicle composition impacts the pH and osmolality of formulations, PBS-base and saline-base were used directly (1× PBS; 1× saline) with the addition of TFV and Q-GRFT. Three formulations (50% PBS, 1× PBS, 1× saline; all with the addition of TFV and Q-GRFT) were chosen to further evaluate the effects of pH adjusting agents on osmolality. For the final screening, both vehicles (PBS-base and saline-base) were diluted into three strengths, 30%, 35%, and 40% with the addition of TFV and Q-GRFT. pH and osmolality were measured as in-line checkpoints for screening and quality assurance.

Manufacturing Procedure: Stock buffer (10× PBS or Saline) was diluted with Milli-Q water to a solution with lower osmolality than desired (˜40 mOsm/kg lower). Then, TFV dry powder was weighed and added into the diluted PBS buffer under constant stirring (250-400 rpm). Under a pH meter, the pH was adjusted to ˜7.0 with 18% NaOH solution, resulting in complete dissolution of TFV. Finally, Q-GRFT stock solution (in 1× PBS) was measured and added to the solution to make the final product. Both PBS- and Saline-based combination enema followed the same procedure. The final solution was assayed for pH, osmolality, and drug content (TFV and Q-GRFT) before division into aliquots.

Stability Study Procedure: Individual enema bottles containing 125 ml of solution were stored under three conditions (4° C.; 25° C./60% RH; 40° C./75% RH) and monitored over time (24 months for 4° C. and 25° C./60% RH; 6 months for 40°° C./75% RH). At each time point (1, 2, 3, 6, 9, 12, 18, and 24 months), physicochemical characteristics were evaluated and compared against the critical quality assessments (CQAs) according to the following procedures.

Appearance: The appearance of the enema solutions was monitored by visual observation of clarity and color. The formulations should be clear and colorless. Any turbidity may indicate the aggregation of Q-GRFT or contaminations, warranting further investigations.

pH: The pH was determined using a pH meter (XL150 pH Benchtop Meters, Fisher Scientific). Before each use, the pH meter was calibrated using standard buffers (Orion All-in-One pH Buffers Kit, Thermo Scientific).

Osmolality: The osmolality of the enema solution was tested using a freeze-point osmometer (Advanced Instruments). About 200 μL of sample solution was injected into the cooling chamber. Then, a supercooled condition (below the freezing temperature) was achieved by the apparatus. While the sample was in the supercooled state, a physical shock was introduced to form a partially crystallized ice-water mixture. The heat of fusion, resulting from the crystallization, raised the sample temperature to a plateau where the solid-liquid equilibrium was maintained. This temperature, representing the true freezing point of the sample, was recorded and calculated to osmolality. Before measurements, a standard osmolality solution (200 mOsm/kg, Advanced Instruments) was used to validate the apparatus. If the deviation was more than ±2 mOsm/kg, the apparatus would be calibrated before testing. For each testing group, the average osmolality values were measured in triplicates.

TFV Content Determination by Ultra Performance Liquid Chromatography: The Ultra Performance Liquid Chromatography (UPLC) method for measuring the TFV content was developed and described previously. For sample preparation, a testing solution was first filtered through a 0.22 μm PTFE filter (Target Syringe Filters, Thermo Scientific) with 1 ml syringes (BD U-100, Fisher Scientific). The samples were diluted 50 times with the mobile phase prior to UPLC analysis. After preparation, samples were quantified by integrating the peak area with the UPLC method (Waters Acquity UPLC H-Class systems; Acquity UPLC BEH C18 columns, 130 Å, 1.7 μm, 2.1×50 mm; with VanGuard Pre-Column, 130 Å, 1.7 μm, 2.1×5 mm), and the TFV content was calculated with calibration curves.

Q-GRFT Content Determination by High-Performance Liquid Chromatography: The High-Performance Liquid Chromatography (HPLC) method for measuring Q-GRFT content was previously developed and reported elsewhere. The samples were diluted 2 times with Milli-Q water. The Q-GRFT content was detected by an HPLC system (Waters Corporation, Milford, MA) equipped with an auto-injector (model 717), a quaternary pump (model 600), and a photodiode array detector (model 2996)) with a C5 column (Jupiter 5 μm 300 Å, 250×4.6 mm) and a C5 pre-column (Security Guard Standard Widepore).

Caco-2 Monolayer Cell Model Toxicity Study Procedure: Passages 33 to 43 of the Caco-2 cell line were used in the studies. The Caco-2 cells were cultured in complete Dulbecco's modified Eagle's medium (DMEM) [500 ml of DMEM (Corning) containing 50 ml fetal bovine serum (FBS; Gibco Qualified One-Shot, Thermo Scientific) and 5 ml PEST (penicillin 10,000 U/ml-streptomycin, 10,000 ug/ml-glutamine 29.2 mg/ml solution (100×); Gibco, Fisher Scientific)] at 37° C. with 5% CO₂ incubation. For the determination of 50% cytotoxic concentrations (CC50), Caco-2 cells were seeded on 96-well plates with 2×10⁴ cells/well. After overnight incubation, the samples were added to the wells and treated for 24 hours. Cell viability was detected using the CellTiter-Glo assay (Promega) with a fluorescence detector. Viability percentage (compared to the untreated groups) was plotted against the concentrations of testing excipients (in log scale). CC50 was calculated using a sigmoidal 4PL model in Prism 9 software. A toxicity study utilizing the Caco-2 model was performed prior to permeability studies to examine the safety of the complete liquid formulations. Samples were applied on the apical side of the Transwell. After 2-hour treatments, the treated samples were discarded and washed off twice with HBSS. A cell viability study was performed with an MTT/isopropanol-extraction method adapted from the literature. TEER values were also measured before and after the treatments as supplemental criteria for the toxicity study.

Caco-2 Monolayer Cell Model Permeability Study Procedure: The procedure was adapted from a previously reported method to build a Caco-2 monolayer cell model and perform the permeability studies. Cell passages 34 to 42 were used to build the model in different batches. For preparation, a total of 3×10⁵ Caco-2 cells were dispensed on the apical side of each Transwell filter. The model would be ready after 21-29 days of culture. The model was validated with a paracellular marker, [¹⁴C]-Mannitol. The apparent permeability (Papp) of [¹⁴C]-Mannitol tested using this monolayer cell model was 3.3×10⁻⁷ cm/s, which was on the same level as the reported value (1.2×10⁻⁷ cm/s). In addition, the cell model was stained with ZO-1 Monoclonal Antibody (ZO1-1A12)-Alexa Fluor 488 (Invitrogen, Thermo Scientific) and observed using a confocal microscope (Nikon AIR confocal microscope). The cell model expressed uniform ZO-1 protein, a tight junction indicator. Z-axis imaging also confirmed that the Caco-2 cells formed a single layer on the substrate. To study the permeability, the sample solution was gently dispensed on the apical side of the model and cultured at 37° C. for 2 hours. At each time point (Time 0, 15, 30, 45, 60, 75, 90, 120 minutes), HBSS samples were collected from the basal sides and tested using UPLC (for TFV) and HPLC (for Q-GRFT). The apparent permeability coefficients (Papp) were calculated using Equation 1, below.

$$Papp = \frac{( \frac{dQ}{dt} )}{A \text{?} C 0} \qquad Equation 1$$

$$\text{?} \text{indicates text missing or illegible when filed}$$

Pₐₚₚ (cm/sec): the apparent permeability coefficient;

$$( \frac{d \text{?}}{\text{?}} ) \qquad Equation 1$$

$$\text{?} \text{indicates text missing or illegible when filed}$$

(ng/sec): the rate of drug transportation; A (cm²): the surface area of the cell layers; C0 (ng/ml): The initial drug concentration in the donor side.

Permeability and Toxicity Assessment with the Ex Vivo Human Colon Tissue: Tissue specimens were collected from patients 18-75 years old. A section of each tissue was retained for histological evaluation with hematoxylin and eosin (H&E) staining assay. After removal of the excess stromal tissue, the epithelial tissue was placed between the donor and receptor compartments of the Ussing chamber apparatus with the epithelial side toward the donor compartment. This set-up was maintained at 37° C. throughout the experiment. Both donor and receptor compartments were filled with KRBG during system calibration. Then the KRBG buffer from the donor sides was replaced with sample solutions. 50 μL was removed from the donor compartment at the beginning and the end of the experiments. At predetermined time points (Time 0, 15, 30, 45, 60, 75, 90, 120 minutes), 200 μL was removed from the receptor chamber and the same volume of fresh KRBG was added for replacement. These samples were held at 4° C. until analyzed by UPLC (for TFV) or HPLC (for Q-GRFT). The Papp values were calculated using Equation 1 as described above. All tissues, including pre-treatment and post-treatment tissues from all groups, were processed for paraffin sectioning. The tissue sections were embedded into paraffin blocks using the Leica EG 1160 embedding station. Tissues were then sectioned at five microns (5 μm) with the Olympus CUT 4060 microtome and placed on slides for H&E staining procedures. Hematoxylin stains the nuclei of cells purple. Eosin stains the other structures of the tissue section red/pink. Microscopy was performed with a Zeiss Axioskop 40 Microscope. Micrographs were obtained with an AxioCam MRc 5 color camera and Axio Vision software. All micrographs were taken with a 10× objective.

Bioactivity Determination using Enzyme-linked Immunosorbent Assay (ELISA): An ELISA was applied to evaluate the gp120 binding activity of select Q-GRFT samples. Nunc MaxiSorp 96-well plates were used for this experiment. The MaxiSorp surface is a hydrophilic/hydrophobic mix that binds to a wide range of biomolecules. In short, gp120 was bound to the wells of a 96-well plate overnight at 4° C. The HIV-1 gp120CM was purchased from Kentucky Bioprocessing (KBP; Part #C-1312). After overnight incubation, the solution of gp120 was removed, and a blocking solution (1× PBS-T [PBS with 0.05% Tween 20]) was applied for two hours at room temperature. After that, the wells were washed and incubated with various dilutions of Q-GRFT samples for one hour. Gp120 binding was detected by applying goat anti-Q-GRFT primary antibody (one-hour incubation) and HRP-labeled rabbit anti-goat secondary antibody (one-hour incubation), sequentially. TMB substrate was applied to the wells after washing of the secondary antibody. Wells were allowed to develop (blue color) for approximately three minutes before the application of sulfuric acid to stop the reaction (yellow color). Gp120 binding was measured at 450 nm.

Statistical Analysis: Each value is reported as means±standard deviation (SD). Statistical data analyses were performed using one-way ANOVA with Tukey's post hoc test, with p<0.05 as the minimal level of significance, p<0.01 for very significant and p<0.001 for highly significant. All tests were performed using the GraphPad Prism software version 9.

### Results

Determination of Manufacturing Procedure: pH and osmolality are two important properties for enema. Therefore, they were measured during the process developments. pH shifted significantly after the addition of TFV and Q-GRFT, an effect which held for both vehicles. In addition, for vehicle-only groups (50% PBS and 1× PBS), osmolalities changed proportionally with the dilutions. After adjusting the pH, osmolalities increased around 20 mOsm/kg for all three testing groups. The targeted osmolality was calculated for the initial vehicle solutions based on the CQAs, dilution factors, and the addition of drugs. The initial osmolality was calculated to be approximately 105 mOsm/kg. A final stage screening was performed for each vehicle with three dilution levels: 30%, 35%, and 40%. Results are summarized in Table 1. For saline-base, 35% saline achieved pH and osmolality criteria. For PBS-base, both 35% and 40% dilutions met the CQAs. Two lead vehicle bases were then chosen, 35% saline and 35% PBS solutions as comparisons.

TABLE 1
The Final Stage (Stage C) Screening Process for
Both Vehicle-based Enema Formulation Development.

| | Initial | | TFV addition | Q-GRFT addition | |
|---|---|---|---|---|---|
| | | Osmolality | & pH adjust- | | Osmolality |
| Group | pH | (mOsm/kg) | ment pH | pH | (mOsm/kg) |
| 30% PBS | 7.57 | 85.0 ± 0.0 | 7.13 | 7.18 | 125.0 ± 1.4 |
| 35% PBS | 7.54 | 97.0 ± 1.4 | 7.35 | 7.28 | 137.5 ± 0.7 |
| 40% PBS | 7.52 | 112.0 ± 0.0 | 7.27 | 7.18 | 137.0 ± 0.0 |
| 30% saline | 6.05 | 83.5 ± 0.7 | 6.95 | 6.98 | 125.0 ± 1.4 |
| 35% saline | 5.82 | 98.5 ± 0.7 | 7.20 | 7.15 | 140.5 ± 0.7 |
| 40% saline | 5.35 | 113.0 ± 0.0 | 7.25 | 7.22 | 153.5 ± 0.7 |

Characterizations of the TFV/Q-GRFT Combination Enema Solutions: Two combination enema solutions with different vehicles (PBS and Saline) were designed, manufactured and monitored in order to provide further options for HIV prevention. Parameters including pH, osmolality, and drug contents of both TFV and Q-GRFT were evaluated. As shown in Table 2 below, all the physiochemical characteristics met the CQAs. Also, no significant difference was observed between the two formulations for all the parameters.

TABLE 2
Physicochemical Characterizations of the
Two Vehicle-based Combination Enema.

| | PBS-base | Saline-base |
|---|---|---|
| CQAs | Combo Enema | Combo Enema |
| Visual: Clear & Colorless | Pass | Pass |
| pH: 6.5-8.0 | 7.12 | 7.08 |
| Osmolality | 139 ± 0.6 | 137 ± 0.6 |
| (mOsm/kg): 145 ± 22 (123-167) | | |
| TFV content [% label claim | 95.2% ± 0.3% | 94.4% ± 1.7% |
| (5.28 mg/ml)]: 90%-110% | | |
| QGRFT content [% label claim | 90.6% ± 4.6% | 93.4% ± 3.5% |
| (0.32 mg/ml)]: 90%-110% | | |

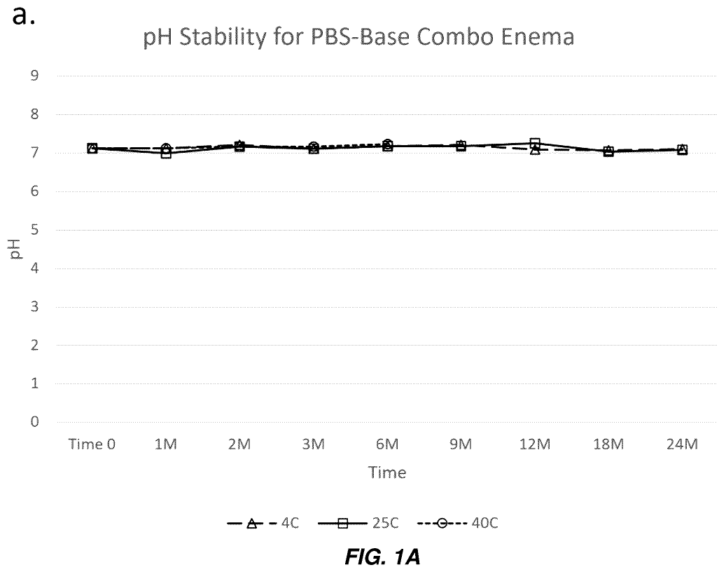

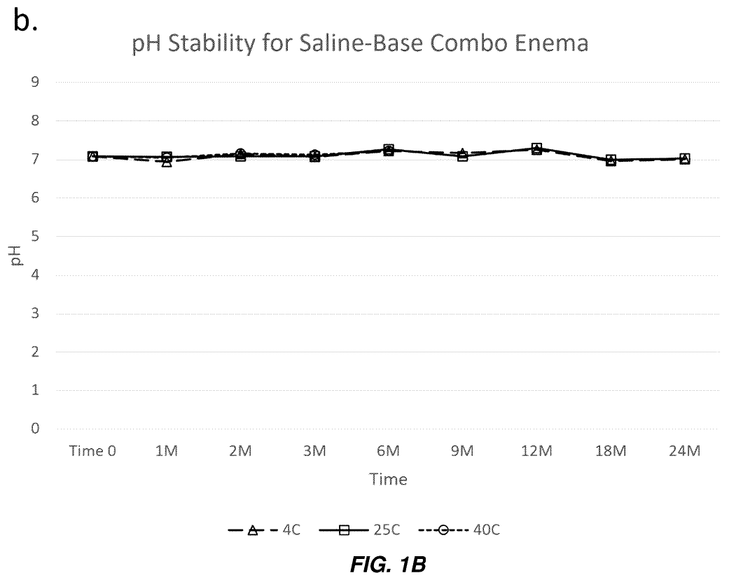

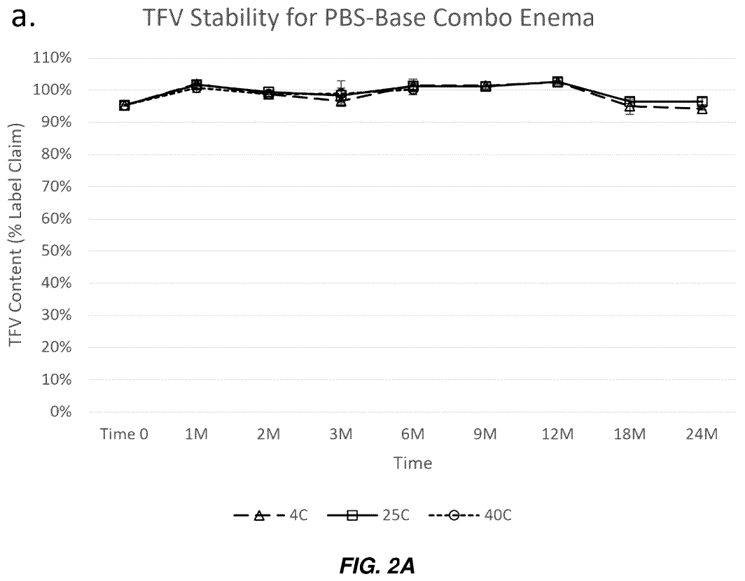

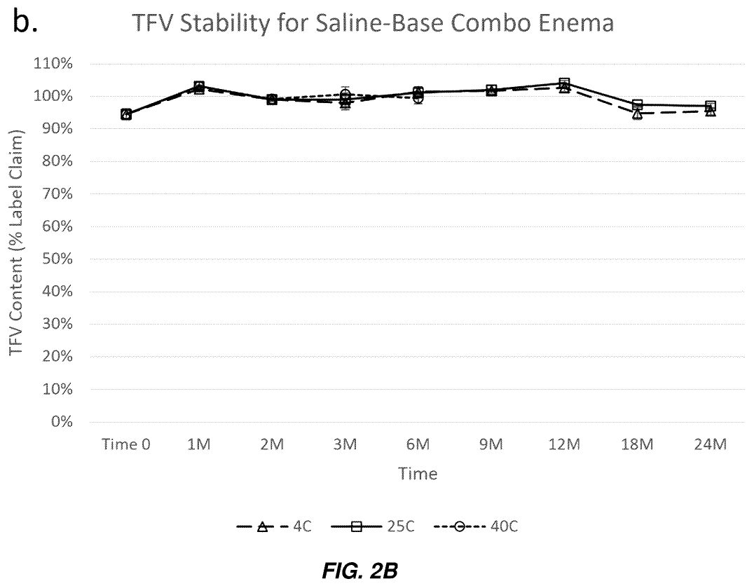

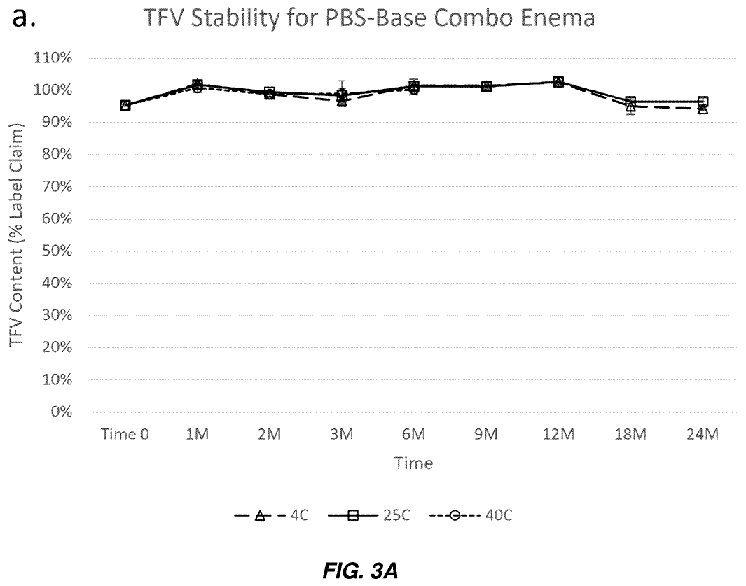

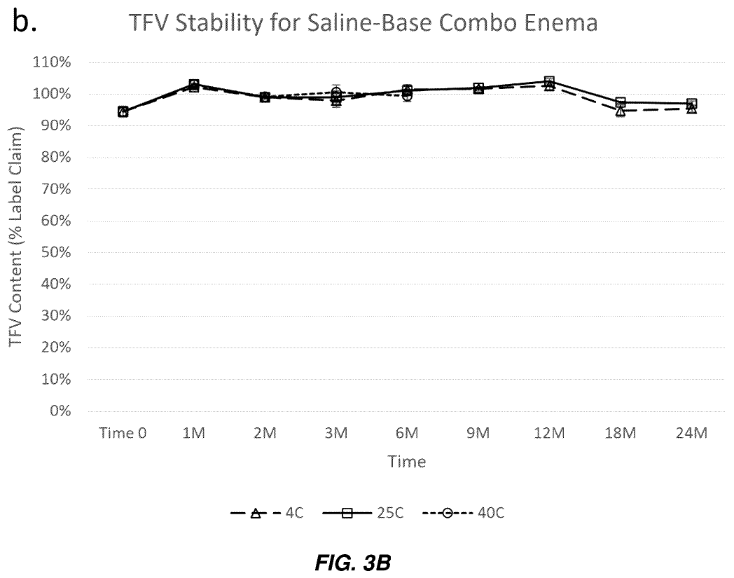

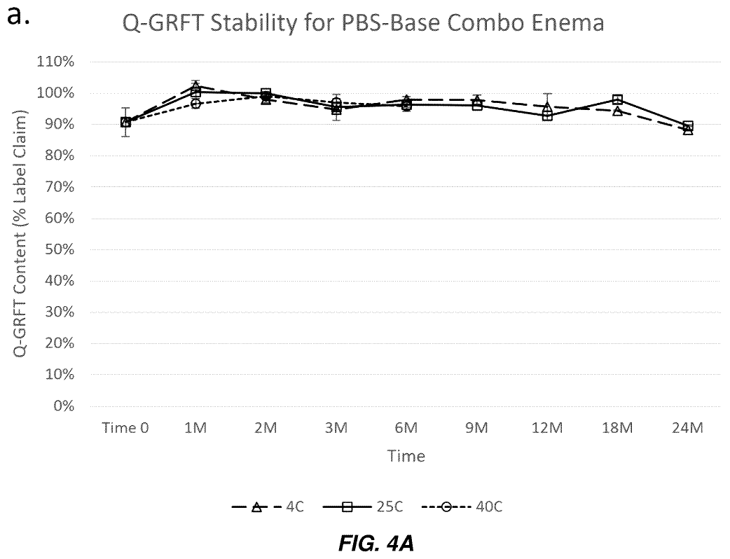

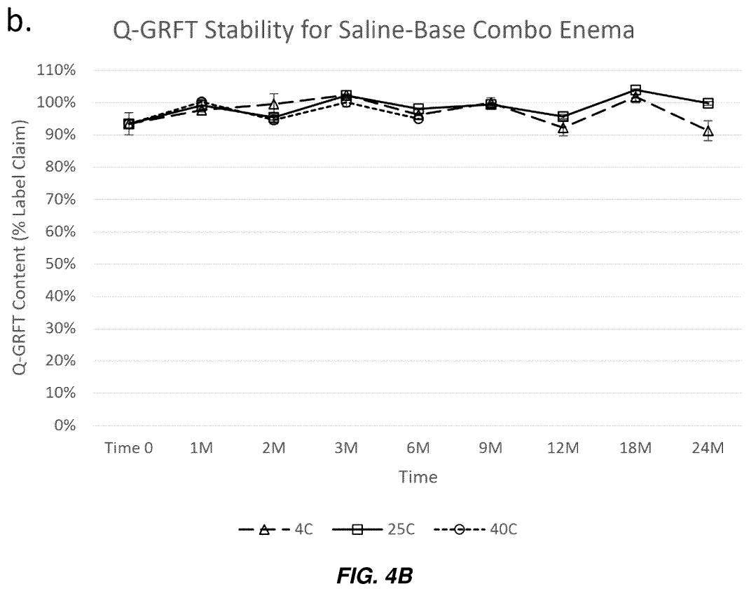

Stability of the Two Combination Enema Formulations: Both enema formulations were dispensed into enema bottles and stored for up to two years under three different conditions: 4° C./60% RH for 24 months, 25° C./60% RH for 24 months, and 40° C./75% RH for 6 months. CQAs were monitored at each time point: 0 months (M), 1M, 2M, 3M, 6M, 9M, 12M, 18M, and 24M. By visual observation, the enema remained clear and colorless throughout the entire stability study. As shown in FIGS. 1A and 1B, pH was also stable for all three conditions. As shown in FIGS. 2A and 2B, both formulations had a small increasing trend with regard to osmolality but remained within the CQA ranges. Nonetheless, both formulations maintained hypotonic (145±22 mOsm/kg) for two years. Drug content is the most important characteristic in this stability study. TFV and Q-GRFT contents were measured by UPLC and HPLC, relatively. For each time point, drug content was compared to the label claim (TFV: 5.28 mg/ml; Q-GRFT: 0.32 mg/ml). As shown in FIGS. 3A and 3B, TFV content remained within range (90%-110% of the label claim) for all time points under all three conditions. Q-GRFT content in the Saline-base formulation also fit in the range for all time points (FIG. 4B). Surprisingly, as shown in FIG. 4A, the PBS-base formulation at 24M had a Q-GRFT content of 89.5% and 88.1% (to the label claim) for the 25° C./60% RH and 4° C. storage conditions, respectively. Although these recovery values were slightly out of the tight range, they were not significantly different from the Q-GRFT content at time 0 months (90.6%).

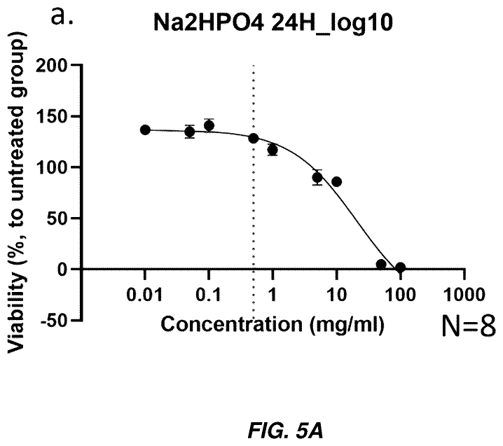

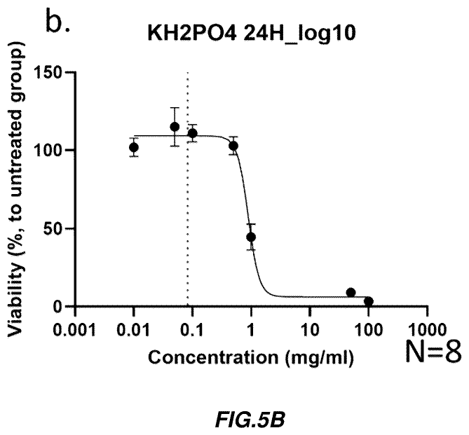

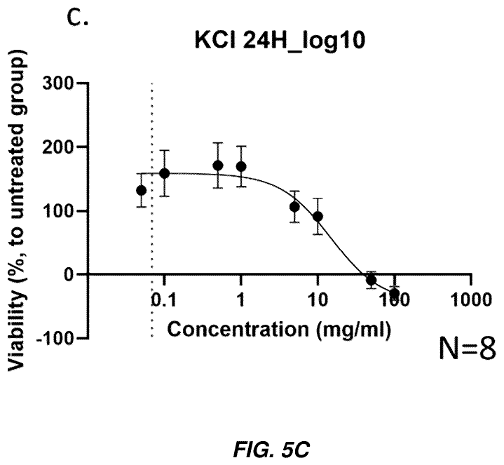

In Vitro Cell Toxicity Studies of the Enema Solutions: Three excipients, namely Na₂HPO₄, KH₂PO₄, and KCl, which have higher concentrations in the combination enema compared to the single-entity enema were selected (highlighted in bold in Table 3). These selected excipients were tested for the CC50 values. For each excipient tested, a series of dilutions was made with culture media and applied to the Caco-2 cells for 24 hours. FIGS. 5A-5C show the representative curves of all selected excipients. At the highest concentrations used in the combination enema formulations (indicated with the red dotted lines), Caco-2 cells remained 100% viability for all three excipients. Furthermore, the CC50 values are significantly higher (for Na₂HPO₄, 40 times higher; for KH₂PO₄, 100 times higher; for KCl, 200 times higher) than the highest concentrations used in the formulations.

TABLE 3
A Composition Comparison of Similar Enema Formulations That Are in Clinical Trials or on the Market (bold
values indicate that the excipient content in the experiment group is higher than in the clinical group).

| | | Experiment Groups | | | | | |
|---|---|---|---|---|---|---|---|
| | | Q-GRFT/ | Q-GRFT/ | | | | |
| | | TFV | TFV | Clinical Groups | | | |
| Content | | Combo | Combo | Q-GRFT | | Commercial Groups | |
| | Ingredients | PBS-base | Saline-base | only | TFV only | Normosol ® | Fleet ® |
| Type | (mg/ml) | (Hypotonic) | (Hypotonic) | (Isotonic) | (Hypotonic) | (Isotonic) | (hypertonic) |
| Active | Q-GRFT | 0.32 | 0.32 | 0.32 | N/A | N/A | N/A |
| Pharmaceutical | | | | | | | |
| Ingredients | TFV | 5.28 | 5.28 | N/A | 5.28 | N/A | N/A |
| (APIs) | | | | | | | |
| Osmolytes | KCl | 0.069 | 0.001 | 0.007 | N/A | N/A | N/A |
| (Salts) | KH₂PO₄ | 0.083 | 0.002 | 0.008 | N/A | N/A | N/A |
| | Na2HPO4 | 0.500 | 0.010 | 0.048 | N/A | N/A | N/A |
| | (anhydrous) | | | | | | |
| | NaCl | 2.777 | 3.117 | 8.966 | 3.3 | 9 | N/A |
| | NaH₂PO₄ | N/A | N/A | N/A | N/A | N/A | 161 |
| | H₂O | | | | | | |
| | Na2HPO4 | N/A | N/A | N/A | N/A | N/A | 59.32 |
| | 7H₂O | | | | | | |
| pH | NaOH | 0.9 | 0.9 | N/A | 1.08 | N/A | pH |
| Modifier | | | | | | | Modifier |

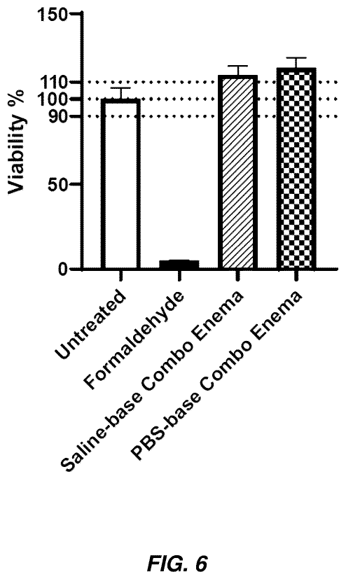

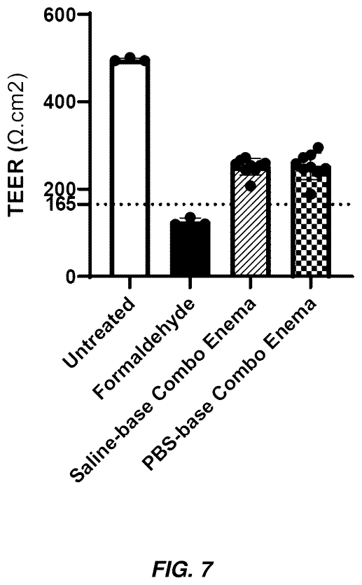

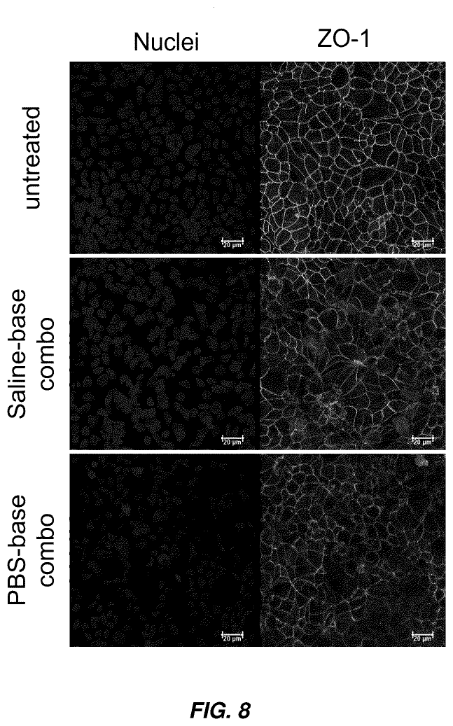

Further evaluation of the entire formulation with full compositions, modifying the Caco-2 monolayer cell model, was performed. The sample liquid was applied on the donor sides while culturing the model in the incubator. Cell viability was measured using an MTT assay with a UV plate reader. As shown in FIG. 6, compared to the untreated group, both of the formulation groups achieved excellent viability (118% for the PBS-base and 114% for the Saline-base). As shown in FIG. 7, TEER values were measured before and after treatments. The positive control group (formaldehyde) had a TEER value below the threshold (165 Ω*cm²). This shows that formaldehyde destroyed the integrity of the cell monolayer and damaged cell viability. In contrast, the two combination enema groups maintained TEER values above the threshold, indicating the integrity of the tight junctions. Evaluation of the cell model after treatments was performed using a confocal microscope. As shown in the confocal images of FIG. 8, wherein the tight junction protein (ZO-1) was stained in green and the nuclei in blue, the cell monolayer maintained its integrity after the 2-hour treatments.

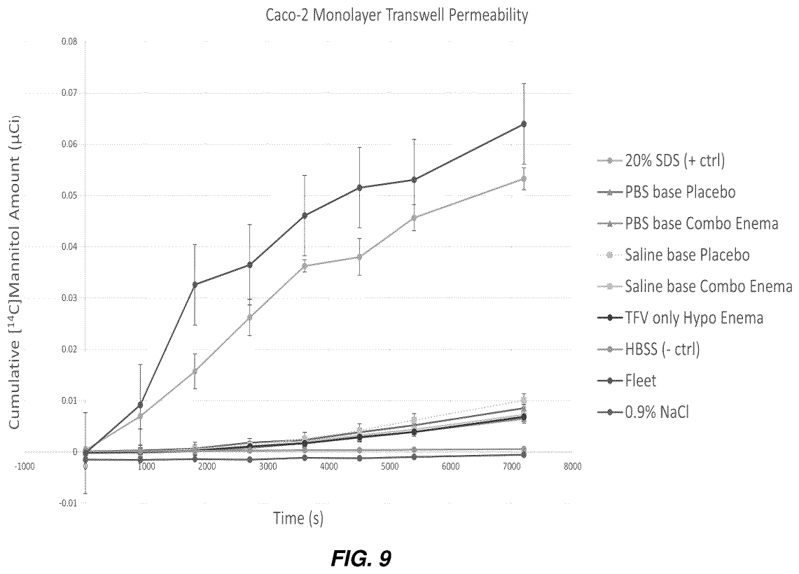

Permeability Validation of the Different Enema Formulations on the Caco-2 Monolayer Cell Model: After the toxicity study, evaluation of the permeability profile using the same Caco-2 monolayer model for enema formulations with different osmolality was performed. Because literature indicated the TFV can penetrate tissues via the paracellular pathway, [¹⁴C]-Mannitol, a paracellular marker, was used to test if different osmolality would result in different permeation profiles. All enema formulations were added with the same concentration of [¹⁴C]-Mannitol and applied on the apical sides of the models for 2 hours. FIG. 9 shows the permeation of [¹⁴C]-Mannitol in all enema formulations used. The apparent permeability values were also calculated and summarized in Table 4. These results demonstrate clearly that these formulations can be divided into three groups based on their permeability profiles.

TABLE 4
A Full Comparison of Multiple Enema Formulations with Different
Osmolality using the Caco-2 Monolayer Cell Model. [¹⁴C]-Mannitol
was used as the paracellular marker and added in all the groups.
The apparent permeability of [¹⁴C]-Mannitol in each
formulation was calculated. N = 3.

| | Paracellular marker [¹⁴C]- | Category¹⁷⁹ |
|---|---|---|
| Group | Mannitol: Papp (cm/s) | Papp (cm/s) |
| HBSS | 2.7 × 10⁻⁷ ± 6.2 × 10⁻⁸ | Poorly absorbed: |
| (negative | | <1 × 10⁻⁶ |
| control) | | |
| 0.9% NaCl | 1.1 × 10⁻⁷ ± 1.5 × 10⁻⁷ | |
| (Normasol ®) | | |
| PBS-base | 4.9 × 10⁻⁶ ± 7.1 × 10⁻⁷ | Moderately absorbed: |
| combo Enema | | 1-10 × 10⁻⁶ |
| PBS-base | 6.7 × 10⁻⁶ ± 1.3 × 10⁻⁶ | |
| Placebo | | |
| Saline-base | 4.8 × 10⁻⁶ ± 3.9 × 10⁻⁷ | |
| combo Enema | | |
| Saline-base | 6.5 × 10⁻⁶ ± 5.7 × 10⁻⁷ | |
| Placebo | | |
| TFV only | 5.3 × 10⁻⁶ ± 7.7 × 10⁻⁷ | |
| Hypo-Enema | | |
| (Clinical | | |
| Group) | | |
| Fleet ® | 2.5 × 10⁻⁵ ± 3.0 × 10⁻⁶ | Well absorbed: |
| 20% SDS | 3.0 × 10⁻⁵ ± 1.0 × 10⁻⁶ | >10 × 10⁻⁶ |
| (positive | | |
| control) | | |

Permeability of TFV Revealed by In Vitro and Ex Vivo Models: After confirming the safety profile, further evaluation of the permeability profile using the same Caco-2 monolayer model was performed. TFV content in the receptor sides was tested for both combination enema as well as the TFV-only Hypo enema, as a comparison. Papp values were calculated and summarized in Table 5. There is no significant difference among all three hypo-osmolar formulations, suggesting that combining with Q-GRFT did not impact the permeability of TFV.

TABLE 5
A Comparison of the Permeability of TFV in Three Hypotonic Enema
Formulations using the Caco-2 Monolayer Cell Model. N = 3-6.

| Hypotonic Enema Formulation | Papp of TFV (cm/s) |
|---|---|
| TFV/Q-GRFT Combo Enema | 1.5 × 10⁻⁶ ± 1.5 × 10⁻⁷ |
| PBS-base (Hypotonic) | |
| TFV/Q-GRFT Combo Enema | 1.8 × 10⁻⁶ ± 2.2 × 10⁻⁷ |
| Saline-base (Hypotonic) | |
| TFV Only Enema (Clinical Group) | 1.8 × 10⁻⁶ ± 2.6 × 10⁻⁷ |
| (Hypotonic) | |

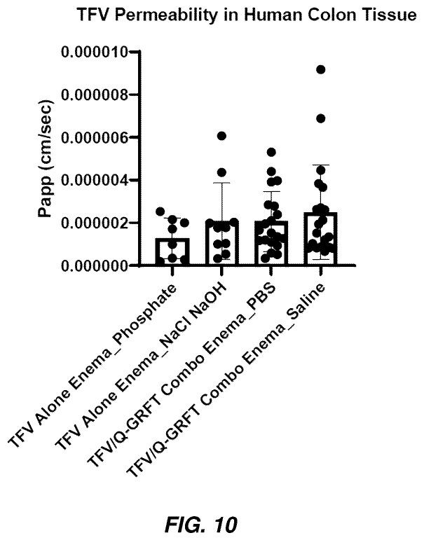

An ex vivo permeability model using human colorectal tissues was also performed to study the permeation of TFV. The calculated Papp of TFV in four hypotonic enema formulations are shown in FIG. 10. Results suggest that there is no significance between any two of the groups. Despite variations, which are commonly seen in human tissue studies, mean Papp values are all similar, indicating that permeation of TFV is independent of formulation composition, including the addition of Q-GRFT.

The Permeability of Q-GRFT Evaluated by the Ex Vivo Colon Tissue Model: Permeation of Q-GRFT was also studied with the ex vivo permeability model. A total of ten tissues from different donors were collected and utilized in this study for the experimental and control groups. The solution from receptor sides was collected at all time points. Donor solution was also collected at the beginning and the end of the experiments. The Q-GRFT content from the receptor sides was lower than the LLOQ of the HPLC method (1 μg/ml) (data not shown). When the donor sides were compared before and after the treatments, the Q-GRFT recovery is 98.69% and 100.72% for PBS- and Saline-base combination enema, respectively (Table 6).

TABLE 6
A Comparison of the Q-GRFT Permeability, Detected
via HPLC, Among Four Formulations in 5 Different
Human Colon Tissue Samples (3-4 replicates each).

| | | Q-GRFT Donor | | |
|---|---|---|---|---|
| | | Side Recovery % | | |
| Tissue | Group | AVG | SD | RSD % |
| Human Colon | TFV/Q-GRFT Combo | 98.69 | 8.53 | 8.64 |
| Tissue | Enema PBS-base | | | |
| | (Hypotonic) | | | |
| | TFV/Q-GRFT Combo | 100.72 | 6.22 | 6.18 |
| | Enema Saline-base | | | |
| | (Hypotonic) | | | |
| Human Cervical | GRFT- | 98.0 | 4.5 | 4.59 |
| Tissue | AlexaFluor488 | | | |
| | (in 1x PBS, Isotonic) | | | |
| | GRFT | 101.3 | 6.8 | 6.71 |
| | (in 1x PBS, Isotonic) | | | |

The toxicity of the combination formulations was evaluated in this model as well. The Ussing Chamber can also monitor the TEER throughout the treatment to confirm the integrity of the epithelium. For the combination formulations, the majority of sample tissues maintained at least 75% of the initial TEER after the 2-hour treatment, suggesting integrity of the colon epithelium. The histology study was also performed using H&E staining. No apparent morphological changes were observed, indicating that both combination formulations caused no toxicity on the human colorectal tissues.

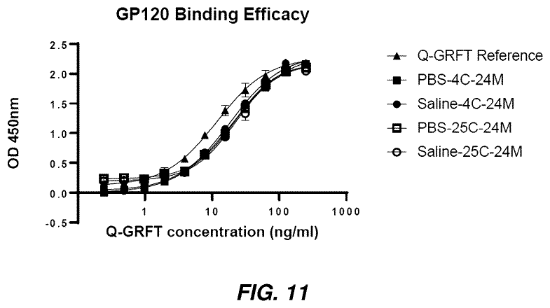

Both Enema Solution Retained Bioactivity after 24 Months: Since Q-GRFT prevents viral entry by binding with the gp120 proteins on the viral surface, the binding efficacy is an important indicator of its bioactivity. Both enema solutions, stored in two conditions, were tested using ELISA. As shown in FIG. 11, the Q-GRFT solution was used as the reference group. The EC50 for the Q-GRFT solution was 11.70 ng/ml. Comparably, the EC50 for the PBS-base combo enema was 20.00 ng/ml (4° C.) and 21.82 ng/ml (25° C./60% RH), and the EC50 for the Saline-base combo enema was 18.39 ng/ml (4° C.) and 22.28 ng/ml (25° C./60% RH). Results from all the groups fit into the target criteria (EC50: 5-50 ng/ml), indicating the stability of Q-GRFT.

### Discussion

As indicated by the U.S. Food and Drug Administration (FDA), the primary goal of patient-focused drug development (PFDD) is to better incorporate the patients' voice in drug development and evaluation. An enema product was selected as the dosage form to provide users with a behaviorally congruent product option for oral PrEP. These on-demand products, incorporating behavioral benefits from the targeted population, i.e. douching among MSM before sex, may potentially offer immediate protection against HIV and other STIs.

A first aim of this study, and the present subject matter, is aimed at providing enema formulations that fulfill patients' needs by identifying the CQAs to the patients' benefits. A second aim was to provide an easy, robust, and reproducible SOP for potential large-scale manufacturing. Preliminary studies with the TFV and Q-GRFT enema indicated that (1) TFV, salts in buffer, and pH adjusting agents are contributors to osmolality, (2) addition of Q-GRFT solution does not change the osmolality significantly in the final product, (3) pH of the solution impacts the solubility of TFV powder, and (4) pH of Q-GRFT stock solution is neutral (around 7.39). pH adjustment can potentially damage the Q-GRFT stability, as instability of GRFT was observed in acidic conditions. Therefore, a simplified procedure of manufacturing the enema solution was developed.

Stability has always been a major issue for liquid formulations containing protein drugs. This study was the first to detect the long-term stability of enema formulations containing Q-GRFT, and the first to explore long-term compatibility of combined TFV and Q-GRFT. As shown in Table 2, the current results showed that both formulations met the CQAs after manufacture. More importantly, physicochemical parameters, including appearance, pH, osmolality, and TFV content, remained stable under three conditions for two years. For Q-GRFT content, the study demonstrated that, despite slight fluctuations, it remained within range for up to 18 months in both formulations. While Q-GRFT content in the PBS-base formulation dropped slightly below 90% at the 24-month time point, these values are still considered stable given that no significant difference was found when compared to the Q-GRFT content at time 0 (90.6%). Overall, the results of this study demonstrated that two stable formulations were achieved. The study provided a successful example of integrating the CQAs into the formulation development process. With the presence of clear and biorelevant CQAs, product quality can be controlled by the release and during the storage.

Toxicity is another important perspective to consider in drug product development. Given the positive safety profile obtained from clinical trials (data unpublished) of the single-entity (TFV-only or Q-GRFT-only) products, the toxicity of the current combo enema with bridge studies was evaluated. As shown in Table 3, a composition comparison for the enema formulations indicates that because the single-entity enema solutions are both based on 1× Saline (0.9% NaCl), the compositions are all similar across these groups. Only three excipients in the PBS-base combo enema have higher concentrations (highlighted in bold in Table 3), compared to the clinical enema formulations. Therefore, Na₂HPO₄, KH₂PO₄, and KCl were tested for their CC50 on Caco-2 cells.

Toxicity of the entire liquid formulations was further evaluated using Caco-2 monolayer cell model. The model was established by seeding Caco-2 cells onto the polycarbonate membranes and cultured until a monolayer of cell was formed. Due to tight junctions formed by the cells, this model can mimic the colorectal epithelium. Overall, results showed that even with higher concentrations, these excipients have no toxicity in Caco-2 cells. Also, results further demonstrated that these two combination enema formulations do not damage the cell viability and integrity in the Caco-2 monolayer model. The formulations of this study have a similar safety profile to the ones in clinical trials. Furthermore, the toxicity studies served as a foundation for the following in vitro permeability study.

Since this Caco-2 monolayer model has been widely used to predict in vivo absorption, this model was used to characterize the formulations with a paracellular marker, [¹⁴C]-Mannitol. Based on the data, the two combination enema formulations, their matching placeboes, and the TFV-only hypo (osmolar) enema (from the clinical trial) can be categorized together. This group has higher [¹⁴C]-Mannitol permeation compared to the HBSS (negative control) and 0.9% NaCl (Normosal®) group, but lower permeation compared to the 20% SDS (positive control) and Fleet® group. As shown in Table 4, these three categories also match the suggested divisions from the literature. It should be noted that both formulations in the well-absorbed group have poor safety profiles, suggesting the high Papp values were achieved due to the damaged epithelium. The HBSS, which was used as a negative control, and the 0.9% NaCl are both iso-osmolar formulations, lacking the driving force for [¹⁴C]-Mannitol permeation into the receptor sides. On the other hand, hypo-osmolar formulations will drive more water into the receptor sides, resulting in higher concentrations of [¹⁴C]-Mannitol, the hydrophilic paracellular marker. Within the moderately absorbed group, all enema formulations have similar Papp values. The data indicates that the paracellular permeability of [¹⁴C]-Mannitol mainly depends on the osmolality of the formulation, regardless of the compositions.

Because TFV is an NRTI, permeability is an important PK parameter. The correlation between pharmacokinetics and pharmacodynamics has already been established 182,183. Results generated from both in vitro and ex vivo models demonstrated good permeation of TFV. By comparing the Papp with the clinical enema (TFV-only enema), both PBS-base and saline-base combo enema achieved desired property. There is no significant difference among all three hypo-osmolar formulations, suggesting that combination with Q-GRFT did not impact the permeability of TFV.

Comparing the Papp of TFV with Papp of [¹⁴C]-Mannitol within each group suggests that TFV achieved similar permeability profiles as [¹⁴C]-Mannitol, indicating that TFV may primarily use the paracellular pathway to penetrate tissues. There is no significant difference between TFV and [¹⁴C]-Mannitol, but the Papp values of TFV are slightly lower. The results echo that TFV is not solely across the tissue via the paracellular pathway. In addition, lower Papp of TFV suggests the potential impacts of transporters. TFV is predominantly transported by MRP4, which is one of the transporters expressed in Caco-2 cells.

Findings from the ex vivo model were also in accordance with the results generated from the in vitro studies. Given that the colon epithelium is also a thin single layer, the results were consistent with that of the Caco-2 monolayer model. Across studies, Papp is generally higher from the ex vivo model than from the in vitro model. This could also be explained by the higher expression of MRP4 in Caco-2 cells than in human intestine tissues, which results in lower Papp values. Hence, the TFV concentration in a patient's tissue may be higher than detected in the in vitro studies.

Q-GRFT was not expected to permeate through the tissues due to its nature as a protein. Literature suggests that Q-GRFT sticks on the surface of vaginal tissue after a 6-hour treatment, but its permeation with regard to colorectal tissues was unknown. With the vaginal epithelium being multi-layered and the colorectal epithelium being single-layered, it was important to evaluate the permeation of Q-GRFT in this study. Preliminary results were compared to demonstrate the location of Q-GRFT. Two differences exist between the studies: (1) vaginal tissues have more epithelial layers than colorectal tissues, and (2) isotonic enema delivers less drug into tissues compared to hypotonic enema. Therefore, if Q-GRFT were permeable, much less recovery from the donor sides would be observed for the hypotonic enema used on the colorectal tissues. Yet the recoveries are all close to 100% across studies, suggesting Q-GRFT would also remain on the surface of the colorectal epithelium, despite the high driving force from hypotonic formulations. In addition, the results included small RSDs, demonstrating low intra- and inter-patient differences.

In summary, this study showed that two clear and hypotonic enema formulations with neutral pH were successfully developed. Both formulations achieved similar permeability of TFV compared to the single entity (TFV-only) enema studied in a clinical trial. The results strongly suggest that Q-GRFT sticks to the surface of colorectal tissues where it will function as an entry inhibitor. In vitro and ex vivo assays demonstrated both formulations to be safe for HIV prevention. Furthermore, this is the first study to demonstrate enema formulations which remain stable for two years. Both products were stable after long-term storage in different conditions, with no significant loss of drug contents of both small molecule and biologic APIs. Moreover, with CQAs incorporated into the design of products, this simple, robust, and reproducible procedure is suitable for future large-scale manufacturing. In summary, these two enema formulations can successfully deliver two APIs with synergistic effects for HIV/STI prevention.

### Example 2

Materials: Recombinant Q-GRFT drug substance was supplied by Kentucky Bioprocessing LLC (Owensboro, KY). Tenofovir (TFV) was sourced from WuXi AppTec (Shanghai, China). PBS 10x molecular biology grade (pH 7.4) was purchased from Mediatech, Inc. (Manassas, VA). Saline buffer (sodium chloride injection, USP, sterile) was purchased from B. Braun Medical Inc. (Irvine, CA). Acetonitrile (ACN), trifluoroacetic acid (TFA), sulfuric acid, t-butylammonium bisulfate (tBAHS), potassium phosphate dibasic (Na₂HPO₄), potassium phosphate monobasic (Na₂HPO₄), potassium chloride (KCl), sodium chloride (NaCl), hydrochloric acid (HCl), sodium hydroxide (NaOH), Sucrose, Trehalose, Lactitol, Maltitol, water content standards (Hydranal), and pH standard kits were obtained from Fisher Scientific (Pittsburgh, PA). The osmolality standards were purchased from Advanced Instruments (Norwood, MA). The protein molecular weight markers (Calbiochem) were purchased from EMD Millipore (Milford, MA). Purified water was prepared in-house utilizing a MilliQ (Millipore; Milford, MA) filtration system at 18.2 MΩ cm. PBS 1× was manufactured in-house with PBS 10× stock and MilliQ-water.

Lyophilization: For samples with cryoprotectants, sugar/sugar polyol powders were weighed and added in individual scintillation vials. The Q-GRFT stock solution was then added. The sample vials were mixed until the powder dissolved. Next, the vials containing sample solution (with or without the addition of cryoprotectants) were transferred to a −80° C. freezer to freeze overnight. Until lyophilization, the samples were tilted and placed in the beaker, attached to a pre-set lyophilizer (Labconco FreeZone Freeze Dry System, Kansas City, MO). The lyophilizing process was set to maintain the environment at −50° C. and 0.070 mBar. Sample vials were measured for their weights after lyophilizing for at least two days. Once the weights were stable, indicating no more water was removed, the lyophilized powder was taken out of the vials and ground with a mortar and pestle. The lyophilized powder would be packed in a tube at 4° C. (unless other conditions were indicated) until further characterizations.

### Physicochemical Characterization of the Lyophilized Powder

Appearance: The appearance of the lyophilized powder was monitored by visual observations. Color, macro particle shape, and texture were recorded.

Crystallinity Determination by Differential Scanning Calorimetry (DSC): Lyophilized powders were characterized for their thermal properties by the Mettler Toledo DSC 3 with the STARe Excellence Software (Columbus, OH). At least 2 mg of powder were measured inside the crucibles to fully cover the bottom surface. Using an empty crucible with a punctured lid as the control, all samples were measured from 25° C. to 250° C. at a heating rate of 10° C./min under a constant nitrogen purge of 20 ml/min. The endothermic peaks were integrated with the built-in software methods.

Crystallinity Determination by X-Ray Powder Diffraction (XRD): Lyophilized powders were tested for crystallization by the Bruker D8 Discover SRD instrument (Billerica, MA) with third-generation Göbel Mirrors to provide maximal X-Ray flux density and an Ultra GID detector. The powders were compressed to form a thin layer on the platform. The measurements start with an angle of 3.5° and end with an angle of 95°. The scan speed is 0.40 seconds/step, and the increment is 0.04°. After scanning, the data was saved using DIFFRACplus BASIC Evaluation software, converted using PowDLL converter software, and graphed using Prism 9.

Flowability: The powder flow was characterized following the Angle of Repose method recorded in the International harmonization of compendial standards chapter <1174>.241. The powder was tested on the Copley BEP2 Flowability Tester (Nottingham, United Kingdom) with a Mitutoyo ABSOLUTE Digimatic Height Gage (Kanagawa, Japan). In short, the testing powder flew through a funnel with a fixed height of 74 mm onto a 100 mm (D, outer diameter) platform. Once the cone-like powder pile was formed, the height of the pile (H) was measured by the gauge. The angle of repose (α) was calculated using Equation 3.1, below. The powder can be classified with different flow properties based on the literature.

$$tan ( α ) = \frac{H}{0.5 * D} \qquad Equation 3.1$$

Size Distribution: The powder was characterized for particle size distribution using a Humboldt HA-4325V motorized sieve shaker with sieve sizes of 1000, 850, 600, 425, 355, 150, 125, 106, 75, 45, and 20 microns (Elgin, IL). The powder was placed in the top sieve (1000 micron) and shaker at level 7 for 30 to 60 minutes. Every sieve was weighed before and after to record the powder weights in each level. Size distribution was later graphed based on the weights of powder.

Water Content: Water content was detected using a Karl-Fischer titration apparatus (Metrohm, 758 KFD Titrino; Herisau, Switzerland). Powders were weighed, transferred to scintillation vials, and cramp-sealed before measuring. The apparatus was set at 120° C. and calibrated with Water Content Standards (Hydranal) each time before the measurements. Water Content (WC %, w/w) was calculated and reported by the software.

### Physicochemical Characterization of the Reconstituted Enema Solution

Appearance: The appearance of the enema solutions was monitored by visual observation. The formulations should be clear and colorless. Any turbidity may indicate the aggregation of Q-GRFT or contaminations, warranting further investigations.

pH: The pH was determined by a pH meter (XL150 pH Benchtop Meters, Thermo Fisher Scientific; Waltham, MA). Before use, the pH meter was calibrated using pH standard buffers (Orion All-in-One pH Buffers Kit, Thermo Fisher Scientific; Waltham, MA) every time. The pH was recorded only after the value stayed stable.

Osmolality: The osmolality assay was performed using a freeze-point osmometer (Advanced Instruments; Norwood, MA). Every time, a standard osmolality solution (200 mOsm/kg, Advanced Instruments; Norwood, MA) was used to test the accuracy of the apparatus. If the deviation was more than ±2 mOsm/kg, a calibration procedure would be performed with a serial range of standard osmolality solutions. For each testing group, the average osmolality values were measured in triplicates.

### Drug Content

TFV Content Determination by Ultra Performance Liquid Chromatography (UPLC): The UPLC method for measuring the TFV content was developed in-house and described previously. For sample preparation, a sample solution was first filtered through a 0.22 μm PTFE filter (Target Syringe Filters, Thermo Fisher Scientific) with 1 ml syringes (BD U-100, Thermo Fisher Scientific). The samples were then diluted 50 times with the mobile phase prior to UPLC analysis. After preparation, samples were quantified by integrating the peak area with the UPLC method (Waters Acquity UPLC H-Class systems; Acquity UPLC BEH C18 columns, 130 Å, 1.7 μm, 2.1×50 mm; with VanGuard Pre-Column, 130 Å, 1.7 μm, 2.1×5 mm), and the TFV content was calculated with calibration curves.

Q-GRFT Content Determination by High-Performance Liquid Chromatography (HPLC): The HPLC method for measuring the Q-GRFT content was developed in-house and reported previously. The samples were diluted 2 times with Milli-Q water. The Q-GRFT content was detected by an HPLC system (Waters Corporation, Milford, MA) equipped with an auto-injector (model 717), a quaternary pump (model 600), and a photodiode array detector (model 2996)) with a C5 column (Jupiter 5 μm 300 Å, 250×4.6 mm) and a C5 pre-column (Security Guard Standard Widepore).

Aggregation Determination by Size Exclusion Chromatography (SEC): An SEC column (TSKgel SuperSW3000 Size Exclusion HPLC column, Tosoh Bioscience) was used for the detection of aggregations. The column was connected to an HPLC system (Thermo Fisher Scientific Dionex UltiMate 3000, Waltham, MA) equipped with an auto-injector, a dual-gradient pump, and an ultraviolet (UV) detector. For sample preparations, the powders were first dissolved in Milli-Q water and then directly injected into the system. The mobile phase is PBS solution at a constant rate (0.3 ml/min). A protein molecular weight marker (Calbiochem, EMD Millipore) was used as a calibration to determine the molecular weights of tested samples.

Bioactivity Determination by Enzyme-linked Immunosorbent Assay (ELISA): An ELISA was adapted and applied to evaluate the gp120 binding activity of the Q-GRFT samples. Nunc MaxiSorp 96-well plates were used for this experiment. The MaxiSorp surface is a hydrophilic/hydrophobic mix that binds to a wide range of biomolecules. In short, gp120 was bound to the wells of a 96-well plate overnight at 4° C. The HIV-1 gp120CM was obtained from Kentucky Bioprocessing (KBP; Part #C-1312). After overnight incubation, the solution of gp120 was removed, and a blocking solution (1× PBS-T [PBS with 0.05% Tween 20]) was applied for two hours at room temperature. After that, the wells were washed and incubated with various dilutions of Q-GRFT samples for one hour. Gp120 binding was detected by applying goat anti-Q-GRFT primary antibody (one-hour incubation) and HRP-labeled rabbit anti-goat secondary antibody (one-hour incubation), sequentially. Tetramethyl benzidine (TMB) substrate was applied to the wells after washing the secondary antibody. Wells were allowed to develop (blue color) for approximately three minutes before the application of sulfuric acid to stop the reaction (yellow color). Gp120 binding was measured at 450 nm with a plate reader.

Statistical Analysis: All values are reported as means +standard deviation (SD). Statistical data analyses were performed using one-way ANOVA with Tukey's post hoc test, with p<0.05 as the minimal level of significance, p<0.01 for very significant, and p<0.001 for highly significant. All statistical analyses were performed using the GraphPad Prism software version 9.

### Results

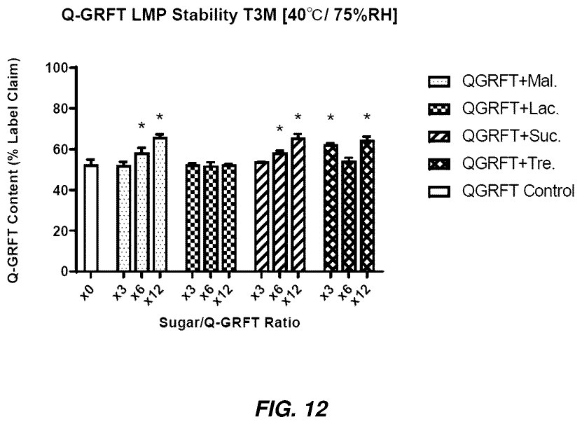

Cryoprotectant Selection Under Accelerated Condition: Because of numerous successes reported by the literature with sugars/sugar polyols, four representatives (sucrose (Suc.), trehalose (Tre.), maltitol (Mal.), and lactitol (Lac.) were included in the study to detect their cryoprotecting effects on Q-GRFT. Q-GRFT solution was mixed with different molar ratios (3, 6, or 12) of sugar/sugar polyol to protein monomer before lyophilization. After lyophilization, the powder was stored in the scintillation vials. The Q-GRFT stability in the lyophilized powder was tested by HPLC after storing in an accelerated condition (40° C./75% RH) for 3 months. As shown in FIG. 12, the protecting effects vary based on the type and the ratio of the sugar/sugar polyols added.

Compared to the control group, which is the Q-GRFT only lyophilized powder group, lactitol has no protecting effects on every ratio. The maltitol and sucrose groups have the same protecting powers, proved by significance higher Q-GRFT content on higher ratios. In addition, this protecting effect is positively correlated with the amounts of sugar/sugar polyol added. For the trehalose group, significantly higher amounts of Q-GRFT content were found in the ratio groups of ×3 and ×12 only. Although a higher amount of Q-GRFT was found in the middle ratio (×6) of the trehalose group, this difference is not statistically significant when compared to the control group.

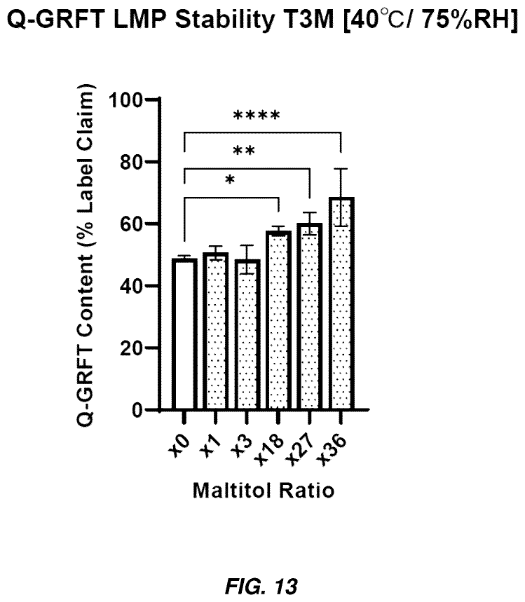

Maltitol Ratio Selections: Given the superiority of maltitol among all four cryoprotectants shown above and in the real-time condition (25° C./60% RH, unpublished data), it was selected as the model protectant for a more detailed ratio selection. To study if the protecting effects remain positively correlated with the amounts of maltitol, Q-GRFT solution was lyophilized with different molar ratios of maltitol to Q-GRFT monomer (1×, 3×, 9×, 18×, 27×, 36×, or 72×). The lyophilized powders were sealed in aluminum pouches, a package proposed for the final product. The pouches were stored under either an acceleration condition (40° C./75% RH) or a real-time condition (25° C./60% RH) for 3 months. As shown in FIG. 13, the results for the accelerated condition showed a similar trend with previous findings. There is a positive correlation between the Q-GRFT content and the ratios of maltitol used in the formulation. The recovery in this study was slightly lower compared to the previous study (for the Q-GRFT only control group), possibly due to different packages. Nonetheless, the Q-GRFT content is significantly higher in the ratio groups of 18×, 27×, and 36×.

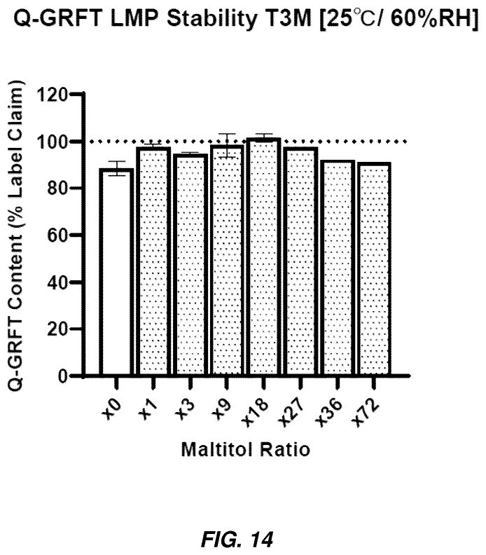

However, the superiority of higher maltitol ratios did not reflect in the normal condition (25° C./60% RH). As shown in FIG. 14, significantly higher Q-GRFT contents were found in all ratio groups except the 36× and 72× groups. Under this condition, the positive trend remains up to 18×. In addition, a higher ratio of maltitol also presented potential difficulties for future productions. Q-GRFT lyophilized powder with higher maltitol contents was stickier due to the hygroscopic nature of maltitol. Therefore, the ratio group of 18× maltitol to Q-GRFT (hereinafter, referred to as the “Q-GRFT lyophilized powder” or “LMP”) was chosen for the following manufacturing and formulation developments.

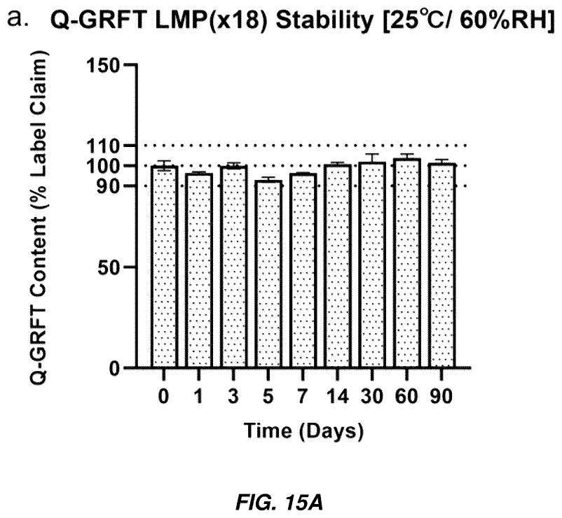

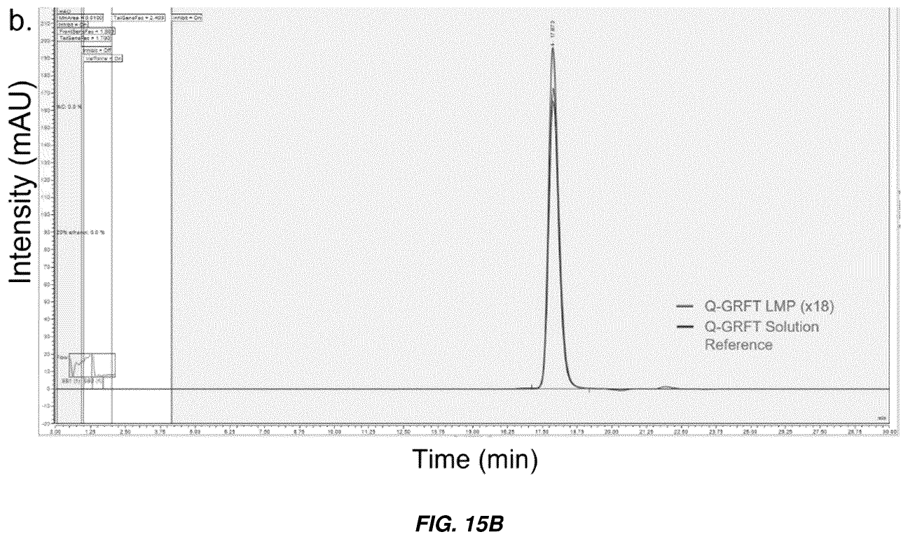

Short Term Stability Study for the Q-GRFT Lyophilized Powder: Since only one single time point (3 months) was investigated in the cryoprotectant selection study, a more detailed short-term study was desired to confirm the stability of Q-GRFT in the real-time condition. The lyophilized Q-GRFT with maltitol powder (LMP) was monitored for Q-GRFT content at several time points (Day 1, 3, 5, 7, 14, 30, 60, 90) in 25° C./60% RH condition. As shown in FIG. 15A, the Q-GRFT content tested was within the range of 90% to 110% for all time points, indicating its stability for up to 3 months. In addition, LMP was also tested in SEC to detect any aggregation. As shown in FIG. 15B, there is no aggregation peak shown on SEC for LMP. In summary, maltitol at a molar ratio of 18 to Q-GRFT was chosen as the cryoprotectant in the formulation. Q-GRFT in the LMP remains stable for up to 3 months and no aggregation was detected.

Physicochemical Characterizations of the Q-GRFT Lyophilized Powder: The LMP formulation was characterized for its physicochemical properties in both solid and reconstituted liquid states. The Q-GRFT only lyophilized powder was also assessed as a comparison. As a pharmaceutical powder, water content (Karl-Fischer), flowability (angle of repose), and crystallinity (DSC and XRD) were measured. The powders were also reconstituted with Milli-Q water to test for osmolality, pH, and drug content, summarized in Table 7. Both lyophilized powders achieved water content below 10% after lyophilization, with slightly higher water in the LMP (8.47%). In addition, flowability was not impacted by the addition of maltitol in the formulation. Both powders achieved angles of repose below 30, which were categorized as excellent flowability.

TABLE 7
A Comparison of Q-GRFT Only Lyophilized Powder and LMP for the Physiochemical Characterizations.
Both the Powder Form and the Reconstituted Enema Form were assessed. N = 3.

| | | | | | | Reconstituted | |
|---|---|---|---|---|---|---|---|
| | Lyophilized Powder | | | | | Enema Solution | |
| | Water | | | | | | Q-GRFT |
| | Content | Flowability | Crystallinity | Crystallinity | Osmolality | | Content (% |
| Formulation | (%) | (Degree) | (Maltilol) | (PBS) | (mOsm/kg) | pH | label claim) |
| Q- | 6.12 ± 1.21 | 26.94 ± 0.52 | N/A | Yes | 0.66 ± 0.58 | 7.23 ± 0.20 | 88.03 ± 2.50 |
| GRFT | | | | | | | |
| only | | | | | | | |
| powder | | | | | | | |
| LMP | 8.47 ± 0.44 | 29.89 ± 0.88 | Partial | No | 0.56 ± 1.01 | 7.30 ± 0.20 | 103.47 ± 7.66 |

In addition, lyophilized powders were reconstituted with Milli-Q water and characterized for osmolality, pH, and drug content. No difference was detected for osmolality and pH between the two groups. However, LMP has higher drug content (103.47% label claim) compared to Q-GRFT only powder (88.03% label claim). Given that the samples were tested at Time 0, the results demonstrated the immediate protecting effects of maltitol to prevent Q-GRFT loss during lyophilization.

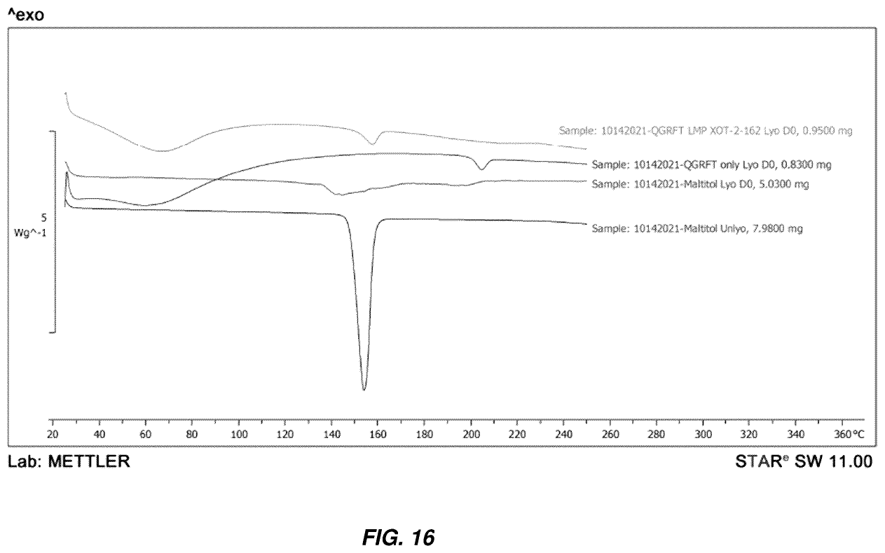

Crystallization Detections by DSC: The crystallinity of lyophilized powders (summarized in Table 7) was tested via DSC, the results of which are shown in FIG. 16. Because the Q-GRFT bulk solution comes in the PBS buffer, PBS exists throughout the entire manufacturing process. Therefore, excipients in PBS (non-lyophilized) were identified by DSC either individually or combined. Only sodium phosphates showed significant endothermic peaks on DSC, with Na₂HPO₄ having a peak of ˜93° C. and NaH₂PO₄ having a peak of about 215° C. These two peaks remained significant even after being combined with NaCl and KCl, despite the predominant existence of NaCl in the formulation (80.97%, w/w to PBS). However, slight decreases were found for both phosphates (for Na₂HPO₄, a decrease of 15° C.; for Na₂HPO₄, a decrease of 3° C.), suggesting interactions between excipients during the heating process. In addition, LMP formulation contains maltitol. Thus, maltitol (before and after lyophilized) powders were also identified by DSC (FIG. 16). Before lyophilization, maltitol crystals present a signature peak on DSC of about 155° C. This signature peak significantly decreased after lyophilization, as partial maltitol transformed into an amorphous state. When Q-GRFT was lyophilized without any cryoprotectants, a significant peak was found around 209° C., indicating the crystallization of PBS (to be specific, Na₂HPO₄). Encouragingly, the PBS crystallization was demolished by the addition of maltitol, with no peak shown near the temperature (FIG. 16). For the LMP group, the endothermic peak shown on DSC at about 158° C. was maltitol. Compared to the maltitol crystal group, this peak is significantly smaller, suggesting excessive maltitol in the LMP formulation exists as a combination of both crystallized and amorphous states.

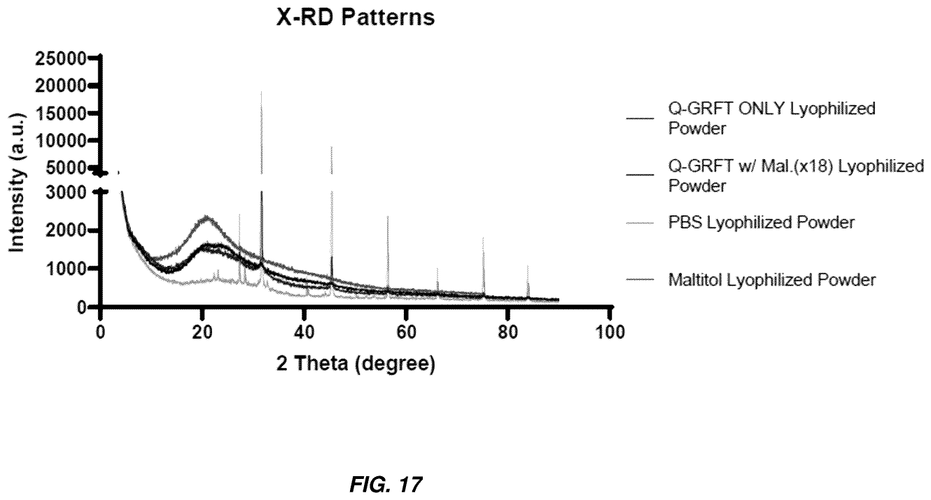

Crystallinity determination by XRD: The crystallinity of lyophilized powders was also confirmed by XRD, the results of which are shown in FIG. 17. PBS was lyophilized and analyzed by XRD, showing some signature peaks. The maltitol-only lyophilized powder did not present any peaks by XRD, confirming its amorphous state (FIG. 17). For the Q-GRFT only lyophilized powder, it has some signature peaks which are identical to PBS. This result indicated crystallization of PBS in the Q-GRFT only group. In the contrast, LMP did not have these peaks, suggesting the addition of maltitol prevented the crystallization of PBS.

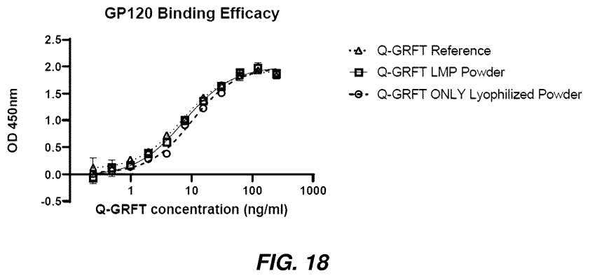

LMP Retained gp120 Binding Bioactivity: The LMP group was reconstituted and tested for gp120 binding efficacy after lyophilization. As shown in FIG. 18, the Q-GRFT solution group was used as the reference. The Q-GRFT reference group had an EC50 of 7.02 ng/ml, while the EC50 of the LMP group was 7.57 ng/ml. In the contrast, the Q-GRFT only lyophilized powder has an EC50 of 10.40 ng/ml. Even though this value fits in the criteria range (5-50 ng/ml), it is higher than the other two groups, suggesting a decrease in gp120 binding efficacy. Results demonstrated that the addition of maltitol during lyophilization (LMP group) can retain the bioactivity of Q-GRFT in powder form.

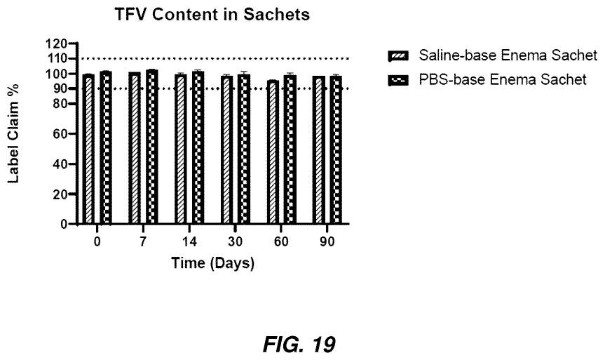

Combination Powder: Short Term Stability Study for the Lyophilized TFV Powders: A combination lyophilized powder formulation was designed to take advantage of the synergetic effects of TFV and Q-GRFT together. After reconstitution, the combination powder can transform into the Q-GRFT/TFV combo enema. A TFV lyophilized powder with two base formulations was developed and monitored for their stability under accelerated conditions. As shown in FIG. 19, TFV content for both formulations was within the 90% to 110% range of label claims. The results showed that lyophilized TFV remained stable for 3 months under the accelerated condition, suggesting a longer shelf life. In addition, TFV was also lyophilized with the addition of maltitol and monitored for short-term stability under the same accelerated condition. Similarly, no drug loss was observed under the accelerated condition for 3 months, demonstrating good compatibility of TFV formulations with maltitol.

Physically Combined Powder Strategy: Since either LMP (lyophilized powder containing Q-GRFT only) or lyophilized TFV powder (both base formulations) has stable drug content individually, a study was performed to determine whether the combined lyophilized powder can achieve all the CQAs after reconstitution. LMP and lyophilized TFV powder were reconstituted with Milli-Q water individually, referred to as Individual Reconstituted Enema). Individual reconstituted enema solutions were combined (referred to as Liquid Combined Enema) to study if there are any interactions post reconstitution. Individual lyophilized powders were also combined first before reconstitution (referred to as Powder Combined Enema) to study if any interactions are involved in the solid form.

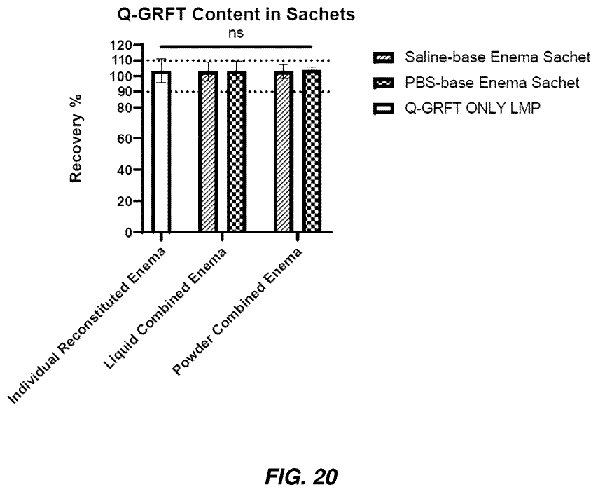

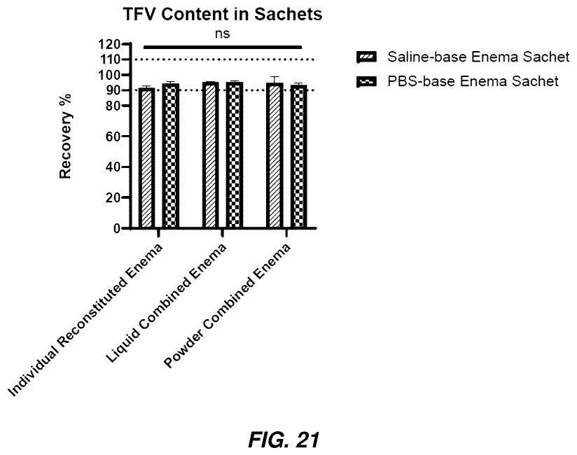

Both Q-GRFT content (FIG. 20) and TFV content (FIG. 21) were determined. For both drugs, all groups achieved drug content within the 90% to 100% range. Moreover, no significant difference was found among groups, indicating no potential drug-drug incompatibility at Time 0. Because pH and osmolality were also important CQAs for enema, these two attributes were also tested for the three groups. All groups demonstrated a pH of around 7. Osmolality was not impacted by combination.

### Discussion

As solid dosage forms offer many advantages over liquid dosage forms, one aim of this study was to develop a solid drug delivery system which can be reconstituted to enema and which will display the enema's characterizations after reconstitution, likely increasing adherence. Therefore, it was needed to develop a stable powder form of an enema sachet. Lyophilization has been demonstrated to be one of the most commonly used processes to prepare dehydrated proteins, including protein purification and formulation developments. Compared to other drying methods, lyophilization is cheaper, and more stable for protein drugs in some cases. However, given the susceptibility of protein drugs, several stresses during the lyophilization process may impact protein stability, including low-temperature stress (cryo-stress), concentration effects, dehydration effects, and pH shifts. Although great efforts have been made in the investigation of protein stabilization during lyophilization, the mechanisms of denaturation are complex. No single theory can elucidate all reasons for protein instability during lyophilization. Excipients were studied by the formulators to stabilize protein drugs, in response to the abovementioned stresses. Literature suggests that by incorporating cryoprotectants, stable protein drugs can be incorporated in the lyophilized formulations.

After studying successful cases, two non-reducing disaccharides (sucrose and trehalose) and two sugar polyols (maltitol and lactitol) were included in the initial screening. In addition, the molar ratio of cryoprotectants to protein drugs plays an important role in stabilization. Therefore, four excipients at three different molar ratios were tested for their protecting powers during lyophilization. As shown in FIG. 12, Q-GRFT content was detected by HPLC and compared against their relative label claims at Time 0. The four tested cryoprotectants presented three different effects: no protection (lactitol), concentration-dependent protection (maltitol and sucrose), and concentration-independent protection (trehalose). The protecting effects observed in this study can be explained by the “water replacement hypothesis”. Sucrose, maltitol, and trehalose, all have eight to nine hydroxyl groups, which offer hydrogen bonds with Q-GRFT. Since the hydro-shell was maintained, the protein's natural conformation should be preserved. In this theory, sugars/sugar polyols served as water substitutes to provide hydrogen bonds, in turn, to prevent aggregation. Surprisingly, this protecting effect was not reflected on lactitol. Lactitol, which also has nine hydroxyl groups, did not stabilize Q-GRFT during lyophilization, possibly due to the spatial differences among the sugar alcohols or intramolecular hydrogen bonds.

Maltitol was selected as the model cryoprotectant for a more in-depth exploration of the protecting effects. As shown in FIG. 13, a more detailed molar ratio panel was investigated for maltitol, echoing the concentration-dependent protecting effects in the accelerated condition. However, when samples were stored and monitored in the real-time condition, the protecting effects were independent of concentrations (FIG. 14). The differences between the two conditions were likely due to the different temperatures. In addition to the “water replacement hypothesis”, recent studies also suggested a “vitrification hypothesis”, where sugars/sugar polyols form a vitrified, rigid sugar-glass matrix that limits the protein degradations kinetically. Maltitol, lyophilized alone has a glass transition temperature (Tg) of 40.6° C. to 43.1° C. The Tg was increased with the addition of proteins or buffers. Results from DSC testing (FIG. 16) and XRD testing (FIG. 17) showed that maltitol stays in an amorphous state in LMP. When the samples were stored in the accelerated condition (40° C./75% RH), which was slightly below the Tg of LMP, annealing or “densification” might occur. Studies found that by heating an amorphous sample below its Tg, a glass will enter the equilibrium glassy state asymptotically, leading to structural relaxation and enhanced protein stability. As maltitol concentration increased, these effects might expand, resulting in the concentration-dependent protections. However, densification is less likely to happen in temperatures far below Tg, which is the real-time condition (25° C./60% RH) in this study. Thus, the concentration dependence was not observed.

Nonetheless, the final lyophilized Q-GRFT powder (LMP) demonstrated good stability in drug content over three months, with no aggregation detected (FIGS. 15A-15B). More importantly, the bioactivity of gp120 binding was maintained for LMP (FIG. 18), suggesting the potential preservation of its HIV-preventing efficacy. Results from DSC testing (FIG. 16) and XRD testing (FIG. 17) also revealed the reason why Q-GRFT was not stable without any cryoprotectants. Without cryoprotectants, maltitol to be specific, PBS buffering agents crystallized in the lyophilized powder. DSC of the Q-GRFT only lyophilized powder showed an endothermic peak (about 209° C.), suggesting the crystallization of NaH₂PO₄. It was also supported by XRD patterns of the Q-GRFT only lyophilized powder (FIG. 17). The Q-GRFT only lyophilized powder group presented some signature peaks, which were the same as the PBS-only lyophilized powder. These results indicated the crystallization of the NaCl, KCl, and NaH₂PO₄. Studies have reported that there was a pronounced reduction in the pH of PBS solution during lyophilization, especially during the cooling process. To be exact, the pH shifted to around 4 because of the crystallization of sodium phosphates. In this acidic condition, the protein drug was not stable, which leads to a decrease in drug content in the Q-GRFT only lyophilized powder. Therefore, it is possible that Q-GRFT was unstable due to the pH shifts caused by the crystallization of PBS.

Consequently, the study offers a third possible explanation for Q-GRFT stability in LMP. Maltitol can inhibit the crystallization of PBS, preventing the Q-GRFT denaturation. As shown in FIGS. 16 and 17, the addition of maltitol diminished the peaks of NaH₂PO₄, NaCl, and KCl, detected by DSC or XRD. Similarly, a recent study reported the reduction of sorbitol crystallization in the presence of maltitol. The findings suggest that maltitol can suppress the crystallization of PBS, leading to the preservation of environmental pH and Q-GRFT stability. This finding is supported by the literature where researchers found that the additions of sugar/sugar polyol preserved the pH in PBS.

Studies for the TFV/Q-GRFT combination lyophilized powder were also included in this work. TFV lyophilized powder formulations were initially designed and developed to achieve similar physiochemical and pharmacodynamic properties as the reconstituted enema. Both of the TFV lyophilized powder formulations (PBS-base and Saline-base) monitored in this study demonstrated good stability in the accelerated condition (FIG. 19) and maltitol compatibility. In the preliminary study, both TFV and Q-GRFT drug contents decreased in the co-lyophilized formulation (unpublished data). It was unexpected to discover the instability of both drugs during lyophilization, suspecting more complex drug-drug interactions or drug-excipient interactions occurred. In order to demolish the denaturation pathways, TFV powder and Q-GRFT powder were incorporated in the solid state, with a goal of improving the compatibility in the physical mixture. After physically mixed, the drug content of both Q-GRFT (FIG. 20) and TFV (FIG. 21) were maintained, with the recovery above 90% for both base formulations (PBS-base and Saline-base). After the combination lyophilized powder was reconstituted, pH and osmolality also fit in the CQAs. The stability study for the physically-combined powder is ongoing. Overall, the physical mixture of the TFV/Q-GRFT combination lyophilized powder showed potential to be utilized as the combo enema.

The LMP also has some limitations that can be investigated in the future. The DSC graph shows that there is an endothermic peak of maltitol in LMP. Since the melting temperature of maltitol is around 149.6° C., results suggested that a small portion of maltitol in the lyophilized formulation crystalized. Possible protein mobility during primary or secondary drying processes can explain the partial crystallinity of maltitol, as similar effects were reported for glucose and sorbitol. It was reported that the crystallization of excipients, including buffer agents and cryoprotectants, may lead to protein drug aggregation. Therefore, the maltitol crystals need to be further monitored and studied. More studies are needed for the combination products in the next stage, including a long-term stability study for the combo powder, bioactivity studies, in vitro and ex vivo permeability studies, and in vivo pharmacokinetic and pharmacodynamic studies for the reconstituted combo enema.

This study aimed to explore the possibility of developing stable Q-GRFT-incorporated lyophilized powder formulations. Cryoprotectants were screened, and the results showed a Q-GRFT lyophilized powder (LMP) which is stable for at least three months in real-time. The LMP also demonstrated acceptable physicochemical properties, both in powder form and in reconstituted liquid form. The gp120 binding efficacy remained after lyophilization. The crystallinity of LMP was further characterized using DSC and XRD. The results revealed a possible cryoprotecting mechanism of maltitol, which is to prevent the crystallization of PBS in the formulations. In addition, the combination powder formulations were further explored with the lyophilized TFV powder. The combination powder demonstrated potential for future development, with drug contents, pH, and osmolality all fitting in CQAs. In summary, the results show successful development of a stable Q-GRFT lyophilized powder formulation and explored the hypothesis of cryoprotection. Although more in vitro, ex vivo, and in vivo studies are needed, the lyophilized powder has the potential to be used either only or combined with other synergetic drugs. The reconstituted enema products can be developed in the future to increase adherence, and in turn, to provide better HIV protection.

Having described this invention, it will be understood to those of ordinary skill in the art that the same can be performed within a wide and equivalent range of conditions, formulations and other parameters without affecting the scope of the invention or any embodiment thereof.

1. A formulation, comprising a therapeutic composition and a griffithsin protein in a composition comprising an excipient and a buffer, wherein the formulation is optionally hypotonic.

2. The formulation of claim 1, wherein the therapeutic composition is an antiretroviral composition, such as a nucleoside reverse transcriptase inhibitor or a nonnucleoside reverse transcriptase inhibitor.

3. (canceled)

4. The formulation of claim 1, wherein the therapeutic composition comprises tenofovir or a pharmaceutically-acceptable salt thereof.

5. (canceled)

6. The formulation of claim 1, wherein the therapeutic composition is tenofovir alefanamide or tenofovir disoproxil.

7. The formulation of claim 1, wherein the composition comprises from about 0.1 mg/ml to about 20 mg/ml, about 1.0 mg/ml to about 10 mg/ml, or about 5.28 mg/ml of the therapeutic composition, and wherein the therapeutic composition is tenofovir.

8. The formulation of claim 1, wherein the griffithsin protein is Griffithsin and/or Q-Griffithsin.

9. The formulation of claim 1, wherein the therapeutic composition comprises from about 0.01 mg/ml to about 10 mg/ml, about 0.1 mg/ml to about 1 mg/ml, about 0.1 mg/ml to about 0.5 mg/ml, or about 0.32 mg/ml of the griffithsin protein.

10. The formulation of claim 1, wherein the therapeutic composition is hypotonic, such as having an osmolality from about 123 mOsm/k to about 332 mOsm/kg, 100 mOsm/kg to about 200 mOsm/kg, about 110 mOsm/kg to about 180 mOsm/kg, about 123 mOsm/kg to about 167 mOsm/kg, about 137 mOsm/kg, about 139 mOsm/kg, or about 145 mOsm/kg and/or a pH from about 6.0 to about 8.5, about 6.5 to about 8.0, or about 7.0.

11. (canceled)

12. (canceled)

13. The formulation of claim 1, wherein the excipient is a disaccharide or sugar polyol, for example, maltitol, lactose, glucose, sorbitol, sucrose, or trehalose.

14. The formulation of claim 1, wherein the buffer is phosphate-buffered saline or 0.9% NaCl saline and/or wherein NaOH, HCl, acetic acid, or citric acid is used to adjust the pH of the therapeutic composition.

15. (canceled)

16. The formulation of claim 1, wherein the therapeutic composition is stable for at least two years.

17. The formulation of claim 1, wherein the formulation is contained in a rectal delivery device, such as sachet for use with a rectal applicator, an enema bag or enema bottle, optionally having an extended tip.

18. A method of producing the formulation of claim 1, comprising:
diluting the buffer and lowering the osmolality to form a first premix;
adding the therapeutic composition to the first premix and mixing until the therapeutic composition is completely dissolved to form a second premix;
adjusting the osmolality or the pH of the second premix;
adding the griffithsin protein to the second premix under constant mixing to form a mixture;
testing the pH or the osmolality of the mixture and, if necessary, adjusting the pH to between 6.5-8 or adjusting the osmolality to between 123 mOsm/kg and 167 mOsm/kg; and
adding the mixture to the rectal delivery device.

19. The method of claim 18, wherein the griffithsin protein is Q-GRFT, and, optionally, the Q-GRFT is provided as a Q-GRFT lyophilized powder prepared by a process comprising dissolving the Q-GRFT lyophilized with a disaccharide or sugar polyol, a disaccharide or sugar polyol, for example, maltitol, lactose, glucose, sorbitol, sucrose, or trehalose, in an aqueous solvent, followed by drying, lyophilizing, or spray-drying the Q-GRFT-containing mixture.

20. A method of treating or preventing a sexually transmitted infection comprising intrarectally delivering the formulation of any one of claim 1 to a patient in a dosage regimen effective to treat or prevent the sexually transmitted infection.

21. The method of claim 20, wherein the sexually transmitted infection is a human immunodeficiency virus (HIV), such as HIV-1 or HIV-2, or a herpes virus, such as herpes simplex virus.

22. (canceled)

23. A method of providing prophylactic protection from human immunodeficiency virus (HIV), comprising placing the formulation of any one of claim 1 intrarectally or orally in a patient.

24. A rectal dosage form, comprising a formulation comprising the composition of any one of claim 1 in the rectal delivery device.

25. The rectal dosage form of claim 24 is configured in a liquid dosage form such as a solution, a suspension, or an emulsion, a solid dosage form such as a suppository, a capsule, a tablet, or a powder form, or a semi-solid dosage form, such as a gel, a foam, or a cream.

26-28. (canceled)

29. A method of producing a stable Q-GRFT lyophilized powder comprising dissolving the Q-GRFT lyophilized with a disaccharide or sugar polyol, a disaccharide or sugar polyol, for example, maltitol, lactose, glucose, sorbitol, sucrose, or trehalose, in an aqueous solvent, followed by drying, lyophilizing, or spray-drying the Q-GRFT-containing mixture.
